(19) United States
(12) Patent Application Publication
(10) Pub. No.: US 2007/0078114 A1
(43) Pub. Date: Apr. 5, 2007

(54) COMBINATION THERAPY FOR ALZHEIMER'S DISEASE AND OTHER DISEASES

(75) Inventors: Adrian Hobden, Salt Lake City, UT (US); Wayne Laslie, Salt Lake City, UT (US)

Correspondence Address: MYRIAD GENETICS INC.;INTELLECUTAL PROPERTY DEPARTMENT, 320 WAKARA WAY, SALT LAKE CITY, UT, 84108, US

(73) Assignee: Myriad Genetics, Incorporated, Salt Lake City, UT (US)

(21) Appl. No.: 11/470,190

(22) Filed: Sep. 5, 2006

Related U.S. Application Data
(60) Provisional application No. 60/713,369, filed on Sep. 2, 2005.

(51) Int. Cl. A61K 31/59; A61K 31/573; A61K 31/192; A61K 31/381

(52) U.S. Cl. 514/167; 514/443; 514/450; 514/570; 514/381; 514/171

(57) ABSTRACT

The invention relates to combinations of one or more Aβ₄₂ lowering agents and one or more hormonal modulating agents.

### CROSS-REFERENCE TO RELATED APPLICATIONS

This application claims priority under 35 U.S.C. § 119(e) to U.S. Provisional Application Ser. No. 60/713,369 filed on Sep. 2, 2005 which is incorporated herein by reference in its entirety.

### FIELD OF THE INVENTION

The invention relates to combinations of steroids and amyloid beta 42 lowering agents for treating diseases.

### BRIEF SUMMARY OF THE INVENTION

The invention provides combinations useful for treating and preventing neurodegenerative disorders including, Alzheimer's disease, mild cognitive impairment, dementia, and other disorders such as cancer.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION OF THE INVENTION

The invention relates to combinations of amyloid beta 42 lowering agents (Aβ₄₂) and hormonal modulating agents. The combinations can be used by patients in need of treatment, for one day or more, one week or more, one month or more, two months or more, three months or more, four months or more, five months or more, six months or more, seven months or more, eight months or more, nine months or more, ten months or more, eleven months or more, twelve months or more, fourteen months or more, sixteen months or more, and eighteen months or more. The combination co-formulations and treatments can be used for neurodegenerative disorders, cancers, and other diseases.

The combination co-formulations can be used for neurodegenerative disorders like Alzheimer's disease. For example, combination treatment with the co-formulations of the invention can provide an improvement, or a lessening of decline, in cognitive function as characterized by cognition tests, measures of global function, activities of daily living, behavior, biochemical disease marker progression, changes in brain volume, and/or plaque pathology to patients in need of such treatment. The cognition tests are those which are capable of measuring cognitive decline in a patient or group of patients. Examples of such tests include cognition tests like the ADAS-cog (Alzheimer's disease Assessment Scale, cognitive subscale) and the MMSE (Mini-Mental State Exam), behavior tests like the NPI (Neuropsychiatric Inventory), daily living activity tests like the ADCS-ADL (Alzheimer's Disease Cooperative Study-Activities of Daily Living), global function test such as the CIBIC-plus (Clinician Interview Based Impression of Change), and CDR sum of boxes (Clinical Dementia Rating).

The Aβ₄₂ lowering agent and hormonal modulating agent combinations can be formulated together in a co-formulation with the active agents combined in one dosage form. The Aβ₄₂ lowering agent and hormonal agent combinations can be co-administered (via a co-formulated dosage form or in separate dosage forms administered at about the same time). The combinations can also be administered separately, at different times, with each agent in a separate unit dosage form. The pharmaceutical compositions of the invention are delivered orally, preferably in a tablet or capsule unit dosage form. The compositions can be used for any therapeutic or prophylactic treatment where the patients benefits from treatment with the combination. For example, the compositions of the invention can be used in methods for treating, preventing, and prophylaxis against neurodegenerative disorders such as Alzheimer's disease.

In a first aspect, the invention provides co-formulations having one or more Aβ₄₂ lowering agents and one or more hormonal modulating agents. This aspect of the invention provides a unit dosage form with one or more Aβ₄₂ lowering agents (and/or pharmaceutically acceptable salts thereof) and one or more hormonal modulating agents (and/or pharmaceutically acceptable salts thereof). In one embodiment, the hormonal modulating agent is chosen from steroids, non-steroids, estrogens, antiandrogens, antiestrogens, progestins, aromatase inhibitors, inhibitors of sex steroid biosynthesis, vitamin D3, vitamin D3 derivatives, vitamin D3 analogues, vitamin D, prolactin secretion inhibitors, and steroidal anti-inflammatory agents. In one embodiment, the hormonal modulating agent is a steroidal anti-inflammatory agent. The composition of this aspect of the invention can be administered orally, once, twice, three, or four times or more per day. When the co-formulation is administered to a patient in need of treatment for one day or more, one week or more, one month or more, two months or more, three months or more, four months or more, five months or more, six months or more, seven months or more, eight months or more, nine months or more, ten months or more, eleven months or more, twelve months or more, fourteen months or more, sixteen months or more, or eighteen months or more, it can provide an improvement or lessening of decline in cognitive function as characterized by cognition tests. The improvement in decline in cognitive function can be more than 20%, 25%, 30%, 35%, 40%, 45%, 50%, 55%, or 60%, as compared to a control. The control can be a plurality of individuals treated with placebo, or may be the expected decline in a test of cognition over a period of time. For example, an individual having probable mild-to-moderate Alzheimer's disease, who is treated with placebo, is expected to score approximately 5.5 points higher (higher scores indicate greater decline on the ADAS-cog) on the ADAS-cog test after a specified period of time of treatment (e.g., 1 year) whereas an individual treated with a composition of the invention for the same period of time will score only about 2.2 points higher on the ADAS-cog scale (a 60% improvement in decline) or only about 3.3 points higher (a 40% improvement in decline in cognitive function). Of course, the actual numeric score will depend upon the test given and the individual. For example, a higher number on the MMSE indicates better cognition, and a lower score (i.e., below 26) indicates some degree of dementia.

Desirably, the oral dose is provided in capsule or tablet form. In a specific embodiment of this aspect of the invention, the dosage is provided as a pharmaceutical composition composed of an Aβ₄₂ lowering agent chosen from (R)-2-(2-fluoro-4-biphenylyl)propionic acid, 5[1-(2-Fluoro-biphenyl-4-yl)-1-methyl-ethyl]-2H-tetrazole, 2-(4-isobutyl-phenyl)-2-methyl propionic acid, 2-(2-fluoro-1,1′-biphenyl-4-yl)-2-methylpropionic acid, and pharmaceutically acceptable salts thereof, and one or more hormonal modulating agents chosen from steroids, non-steroids, estrogens, antiandrogens, antiestrogens, progestins, aromatase inhibitors, inhibitors of sex steroid biosynthesis, vitamin D3, vitamin D3 derivatives, vitamin D3 analogues, vitamin D, prolactin secretion inhibitors, and steroidal anti-inflammatory agents. In a specific embodiment of this aspect of the invention, the dosage is provided as a pharmaceutical composition composed of an Aβ₄₂ lowering agent chosen from (R)-2-(2-fluoro-4-biphenylyl)propionic acid, 5[1-(2-Fluoro-biphenyl-4-yl)-1-methyl-ethyl]-2H-tetrazole, 2-(4-isobutyl-phenyl)-2-methyl propionic acid, and 2-(2-fluoro-1,1′-biphenyl-4-yl)-2-methylpropionic acid, and one or more steroidal anti-cancer agents chosen from 1,25-dihydroxyvitamin D3, 1,25-dihydroxyvitamin D3 derivatives, antiandrogens, antiestrogens, aromatase inhibitors, progestins, and prolactin secretion inhibitors. In a specific embodiment of this aspect of the invention, the dosage is provided as a pharmaceutical composition composed of an Aβ₄₂ lowering agent chosen from (R)-2-(2-fluoro-4-biphenylyl)propionic acid, 5[1-(2-fluoro-biphenyl-4-yl)-1-methyl-ethyl]-2H-tetrazole, 2-(4-isobutyl-phenyl)-2-methyl propionic acid, 2-(2-fluoro-1,1′-biphenyl-4-yl)-2-methylpropionic acid, and pharmaceutically acceptable salts thereof, and one or more steroidal anti-inflammatory agents chosen from budesonide, pregnenolone, prednisone, prednisolone, methylprednisolone, triamcinolone, dexamethasone, betamethasone, parametasone, cortisone, and hydrocortisone. In another specific embodiment of this aspect of the invention, the dosage is provided as a pharmaceutical composition composed of (R)-2-(2-fluoro-4-biphenylyl)propionic acid and one or more steroidal anti-inflammatory agents chosen from budesonide, pregnenolone, prednisone, prednisolone, methylprednisolone, triamcinolone, dexamethasone, betamethasone, parametasone, cortisone, and hydrocortisone. The unit dosage form of this aspect of the invention has one or more pharmaceutically acceptable excipients.

In a related aspect, the invention provides a method of treating an individual having, or suspected of having, Alzheimer's disease, comprising administering, to an individual in need of treatment, a combination of one or more Aβ₄₂ lowering agents (and/or pharmaceutically acceptable salts thereof) and one or more steroidal anti-inflammatory agents (and/or pharmaceutically acceptable salts thereof). The invention further provides a method of improving a decline in a measure of cognitive function of an individual comprising administering a combination of one or more Aβ₄₂ lowering agents and one or more steroidal anti-inflammatory agents to the individual, where the administration of the combination results in a reduction of the decline in the measure of cognitive function as compared to a control. In another specific embodiment, the reduction in the decline in the measure of cognitive function is 25% or more compared to the control. In another specific embodiment, the reduction in decline in the measure of cognitive function is 40% or more compared to the control. In another specific embodiment, the improvement in the decline in the measure of cognitive function is 60% or more compared to the control. In another specific embodiment, the measure of cognitive function is an ADAS-cog test. In a specific embodiment, the reduction in decline is about 2.2 points or less in the ADAS-cog test over one year. In another specific embodiment, the reduction in decline is about 3.3 points or less in the ADAS-cog test over one year. In one embodiment, the Aβ₄₂ lowering agent is (R)-2-(2-fluoro-4-biphenylyl)propionic acid or a pharmaceutically acceptable salt thereof . In a specific embodiment, the (R)-2-(2-fluoro-4-biphenylyl)propionic acid is administered in a dose of about 800 or more mg twice daily. In another specific embodiment, the (R)-2-(2-fluoro-4-biphenylyl)propionic acid is administered in a dose of about 1000 mg or more twice daily. In one specific embodiment, the one or more steroidal anti-inflammatory agents are chosen from budesonide, pregnenolone, prednisone, prednisolone, methylprednisolone, triamcinolone, dexamethasone, betamethasone, parametasone, cortisone, and hydrocortisone.

In another related aspect, the invention provides for a method of improving or lessening the rate of decline in a disease biomarker (i.e., reversing or slowing the progression of), in an individual having, or suspected of having, Alzheimer's disease, comprising administering a combination of one or more Aβ₄₂ lowering agents and one or more steroidal anti-inflammatory agents to the individual. Disease progression may be monitored by one or more Alzheimer's disease markers. In a specific embodiment, the administration is continued once a day or more, for four months or more. In another specific embodiment, the administration is continued once a day or more, for one day or more, one week or more, one month or more, two months or more, three months or more, four months or more, five months or more, six months or more, seven months or more, eight months or more, nine months or more, ten months or more, eleven months or more, twelve months or more, fourteen months or more, sixteen months or more, or eighteen months or more. In a specific embodiment, the disease marker is amyloid beta peptide (Aβ), Aβ₄₂, Aβ₄₀, Aβ₄₂/Aβ₄₀ ratios, ratios of Aβ peptides, and/or tau. In a specific embodiment, the Aβ₄₂ lowering agent is (R)-2-(2-fluoro-4-biphenylyl)propionic acid or a pharmaceutically acceptable salt thereof. In another specific embodiment, (R)-2-(2-fluoro-4-biphenylyl)propionic acid is administered in a dose of about 800 or more mg twice daily. In another specific embodiment, the (R)-2-(2-fluoro-4-biphenylyl)propionic acid is administered in a dose of about 1000 or more mg twice daily. In a specific embodiment of this aspect of the invention, one or more Aβ₄₂ lowering agents are chosen (R)-2-(2-fluoro-4-biphenylyl)propionic acid, 5[1-(2-fluoro-biphenyl-4-yl)-1-methyl-ethyl]-2H-tetrazole, 2-(4-isobutyl-phenyl)-2-methyl propionic acid, 2-(2-fluoro- 1,1 ′-biphenyl-4-yl)-2-methylpropionic acid, and pharmaceutically acceptable salts thereof, and one or more steroidal anti-inflammatory agents are chosen from budesonide, pregnenolone, prednisone, prednisolone, methylprednisolone, triamcinolone, dexamethasone, betamethasone, parametasone, cortisone, and hydrocortisone. In another specific embodiment of this aspect of the invention, the dosage is provided as a pharmaceutical composition composed of (R)-2-(2-fluoro-4-biphenylyl)propionic acid and one or more steroidal anti-inflammatory agents chosen from budesonide, pregnenolone, prednisone, prednisolone, methylprednisolone, triamcinolone, dexamethasone, betamethasone, parametasone, cortisone, and hydrocortisone. In one specific embodiment, the one or more steroidal anti-inflammatory agents are chosen from budesonide, pregnenolone, prednisone, prednisolone, methylprednisolone, triamcinolone, dexamethasone, betamethasone, parametasone, cortisone, and hydrocortisone.

In a third aspect, the invention provides a method of improving, or lessening a decline in, plaque pathology associated with Alzheimer's disease in an individual having, or suspected of having, Alzheimer's disease, comprising administering a combination of one or more Aβ₄₂ lowering agents (and/or pharmaceutically acceptable salts thereof) and one or more steroidal anti-inflammatory agents to the individual (and/or pharmaceutically acceptable salts thereof). In a specific embodiment, the administration is continued once a day or more for four months or more. In another specific embodiment, the administration is continued once a day or more, for one day or more, one week or more, one month or more, two months or more, three months or more, four months or more, five months or more, six months or more, seven months or more, eight months or more, nine months or more, ten months or more, eleven months or more, twelve months or more, fourteen months or more, sixteen months or more, or eighteen months or more. In a specific embodiment of this aspect of the invention, the Aβ₄₂ lowering agent is (R)-2-(2-fluoro-4-biphenylyl)propionic acid. In another specific embodiment, (R)-2-(2-fluoro-4-biphenylyl)propionic acid is administered in a dose of about 800 or more mg twice daily. In another specific embodiment, (R)-2-(2-fluoro-4-biphenylyl)propionic acid is administered in a dose of about 1000 or more mg twice daily. In a specific embodiment of this aspect of the invention, the one or more Aβ₄₂ lowering agents are chosen (R)-2-(2-fluoro-4-biphenylyl)propionic acid, 5[1-(2-Fluoro-biphenyl-4-yl)-1-methyl-ethyl]-2H-tetrazole, 2-(4-isobutyl-phenyl)-2-methyl propionic acid, 2-(2-fluoro-1,1′-biphenyl-4-yl)-2-methylpropionic acid, and pharmaceutically acceptable salts thereof, and one or more steroidal anti-inflammatory agents are chosen from budesonide, pregnenolone, prednisone, prednisolone, methylprednisolone, triamcinolone, dexamethasone, betamethasone, parametasone, cortisone, and hydrocortisone. In another specific embodiment of this aspect of the invention, the dosage is provided as a pharmaceutical composition composed of (R)-2-(2-fluoro-4-biphenylyl)propionic acid and one or more steroidal anti-inflammatory agents chosen from budesonide, pregnenolone, prednisone, prednisolone, methylprednisolone, triamcinolone, dexamethasone, betamethasone, parametasone, cortisone, and hydrocortisone. In one specific embodiment, the one or more steroidal anti-inflammatory agents are chosen from budesonide, pregnenolone, prednisone, prednisolone, methylprednisolone, triamcinolone, dexamethasone, betamethasone, parametasone, cortisone, and hydrocortisone.

In a fourth aspect, the invention provides a method of treating Alzheimer's disease comprising administering to a patient in need of such treatment, a pharmaceutical composition comprising an effective amount of a combination of one or more Aβ₄₂ lowering agents (and/or pharmaceutically acceptable salts thereof) and one or more steroidal anti-inflammatory agents (and/or pharmaceutically acceptable salts thereof). Oral administration of the composition of this aspect of the invention twice daily for one day or more, one week or more, one month or more, two months or more, three months or more, four months or more, five months or more, six months or more, seven months or more, eight months or more, nine months or more, ten months or more, eleven months or more, twelve months or more, fourteen months or more, sixteen months or more, or eighteen months or more, it can provide an improvement or lessening in decline of cognitive function as characterized by cognition tests, biochemical disease marker progression, and/or plaque pathology. Desirably, the oral dose is provided in capsule or tablet form. In a specific embodiment of this aspect of the invention, the Aβ₄₂ lowering agent is (R)-2-(2-fluoro-4-biphenylyl)propionic acid. According to a specific embodiment of this aspect of the invention, a patient in need of treatment is administered an Alzheimer's disease treating effective amount of a pharmaceutical composition having (R)-2-(2-fluoro-4-biphenylyl)propionic acid, one or more pharmaceutically excipients, and one or more steroidal anti-inflammatory agents chosen from budesonide, pregnenolone, prednisone, prednisolone, methylprednisolone, triamcinolone, dexamethasone, betamethasone, parametasone, cortisone, and hydrocortisone. In a specific embodiment of this aspect of the invention, the one or more Aβ₄₂ lowering agents are chosen (R)-2-(2-fluoro-4-biphenylyl)propionic acid, 5[1-(2-Fluoro-biphenyl-4-yl)-1-methyl-ethyl]-2H-tetrazole, 2-(4-isobutyl-phenyl)-2-methyl propionic acid, 2-(2-fluoro-1,1′-biphenyl-4-yl)-2-methylpropionic acid, and pharmaceutically acceptable salts thereof, and one or more steroidal anti-inflammatory agents are chosen from budesonide, pregnenolone, prednisone, prednisolone, methylprednisolone, triamcinolone, dexamethasone, betamethasone, parametasone, cortisone, and hydrocortisone. In another specific embodiment of this aspect of the invention, the dosage is provided as a pharmaceutical composition composed of (R)-2-(2-fluoro-4-biphenylyl)propionic acid and one or more steroidal anti-inflammatory agents chosen from budesonide, pregnenolone, prednisone, prednisolone, methylprednisolone, triamcinolone, dexamethasone, betamethasone, parametasone, cortisone, and hydrocortisone. The method of this aspect of the invention involves identifying individuals likely to have mild-to-moderate Alzheimer's disease. Individuals having probable mild-to-moderate Alzheimer's disease can be diagnosed by any method available to the ordinary artisan skilled in such diagnoses. For example, diagnosis can be according to DSM-IV (TR) and/or meets NINCDS-ADRDA criteria for probable AD. According to a specific embodiment of this aspect of the invention, individuals with probable mild-to-moderate AD can take an oral dose of a pharmaceutical composition having one or more Aβ₄₂ lowering agents and one or more steroidal anti-inflammatory agents (e.g., twice daily dosing with two tablets containing 400 mg of (R)-2-(2-fluoro-4-biphenylyl)propionic acid and one tablet containing 10 mg prednisone) for one day or more, one week or more, one month or more, two months or more, three months or more, four months or more, five months or more, six months or more, seven months or more, eight months or more, nine months or more, ten months or more, eleven months or more, twelve months or more, fourteen months or more, sixteen months or more, or eighteen months or more. Individuals undergoing such treatment are likely to see an improvement or lessening in decline of cognitive function, an improvement or lessening in decline in biochemical disease marker progression, and/or an improvement or lessening in decline in plaque pathology. A lessening in decline in cognitive function can be assessed using test of cognitive function like the ADAS-cog. For example, an individual treated with placebo having probable mild-to-moderate Alzheimer's disease is expected to score approximately 5.5 points higher on the ADAS-cog test after a specified period of time of treatment (e.g., 1 year) whereas an individual treated with the composition of this aspect of the invention for the same period of time will score approximately 2.2 points higher on the ADAS-cog scale, i.e., a 60% decrease in decline, or 3.3 points higher, i.e., a 40% decrease in decline in cognitive function. In one embodiment, the individual in need of treatment has mild Alzheimer's disease. In another embodiment, the individual in need of treatment has mild cognitive embodiment.

In a fifth aspect, the invention provides a method of preventing the onset of Alzheimer's disease comprising administering to a patient in need of or desiring such treatment, a combination of one or more Aβ₄₂ lowering agents (and/or pharmaceutically acceptable salts thereof) and one or more steroidal anti-inflammatory agents (and/or pharmaceutically acceptable salts thereof). Oral administration of a combination of one or more Aβ₄₂ lowering agents and one or more steroidal anti-inflammatory agents for one day or more, one week or more, one month or more, two months or more, three months or more, four months or more, five months or more, six months or more, seven months or more, eight months or more, nine months or more, ten months or more, eleven months or more, twelve months or more, fourteen months or more, sixteen months or more, or eighteen months or more, delays the onset of decline of cognitive function, biochemical disease marker progression, and/or plaque pathology. According to one aspect of this embodiment, an individual desiring or needing preventative treatment against the onset of AD is administered twice daily a dose having from about 400 mg to about 800 mg of (R)-2-(2-fluoro-4-biphenylyl)propionic acid. Desirably, the oral dose is provided in capsule or tablet form. The preventive treatment is preferably maintained as long as the individual continues to desire or need the treatment. Individuals needing or desiring preventative treatment against AD can be those having risk factors for developing AD. For example, risk factors for developing AD can be genetic factors or environmental factors. In one embodiment, the risk factor is age. Genetic risk factors can be assessed in a variety of ways, such as ascertaining the family medical history of the individual, or performing a genetic test to identify genes that confer a predisposition for developing AD. Additionally, risk factors can be assessed by monitoring genetic and biochemical markers. In a specific embodiment of this aspect of the invention, one or more Aβ₄₂ lowering agents are chosen (R)-2-(2-fluoro-4-biphenylyl)propionic acid, 5[1-(2-Fluoro-biphenyl-4-yl)-1-methyl-ethyl]-2H-tetrazole, 2-(4-isobutyl-phenyl)-2-methyl propionic acid, 2-(2-fluoro-1,1′-biphenyl-4-yl)-2-methylpropionic acid, and pharmaceutically acceptable salts thereof, and one or more steroidal anti-inflammatory agents are chosen from budesonide, pregnenolone, prednisone, prednisolone, methylprednisolone, triamcinolone, dexamethasone, betamethasone, parametasone, cortisone, and hydrocortisone. In another specific embodiment of this aspect of the invention, the dosage is provided as a pharmaceutical composition composed of (R)-2-(2-fluoro-4-biphenylyl)propionic acid and one or more steroidal anti-inflammatory agents chosen from budesonide, pregnenolone, prednisone, prednisolone, methylprednisolone, triamcinolone, dexamethasone, betamethasone, parametasone, cortisone, and hydrocortisone. In one specific embodiment, the one or more steroidal anti-inflammatory agents are chosen from budesonide, pregnenolone, prednisone, prednisolone, methylprednisolone, triamcinolone, dexamethasone, betamethasone, parametasone, cortisone, and hydrocortisone.

In a sixth aspect, the invention provides a method of decelerating the onset of Alzheimer's disease comprising administering to a patient in need of such treatment, a pharmaceutical composition comprising an effective amount of a combination of one or more Aβ₄₂ lowering agents (and/or pharmaceutically acceptable salts thereof) and one or more steroidal anti-inflammatory agents (and/or pharmaceutically acceptable salts thereof). Oral administration of a combination of one or more Aβ₄₂ lowering agents and one or more steroidal anti-inflammatory agents for one day or more, one week or more, one month or more, two months or more, three months or more, four months or more, five months or more, six months or more, seven months or more, eight months or more, nine months or more, ten months or more, eleven months or more, twelve months or more, fourteen months or more, sixteen months or more, or eighteen months or more provides a deceleration in decline of cognitive function, biochemical disease marker progression, and/or plaque pathology. According to this aspect of the invention, an individual having mild cognitive impairment that is likely progress to AD is identified. Alternatively, the individual can be in the prodromal stage of AD development. Upon identification of an individual having mild cognitive impairment likely to progress to Alzheimer's disease or being in the prodromal stage of AD development, a preventive treatment regimen is prescribed for the patient. The preventive treatment regimen involves administering to the individual in need or desiring such treatment a pharmaceutical composition sufficient to decelerate the onset of Alzheimer's disease. The composition for use in this aspect of the invention is designed in such as to be suitable for chronic long-term use with a prophylactic effect. In a specific embodiment of this aspect of the invention, the Aβ₄₂ lowering agent is (R)-2-(2-fluoro-4-biphenylyl)propionic acid or a pharmaceutically acceptable salt thereof. In a specific embodiment of this aspect of the invention, the one or more Aβ₄₂ lowering agents are chosen (R)-2-(2-fluoro-4-biphenylyl)propionic acid, 5[1-(2-Fluoro-biphenyl-4-yl)-1-methyl-ethyl]-2H-tetrazole, 2-(4-isobutyl-phenyl)-2-methyl propionic acid, 2-(2-fluoro-1,1′-biphenyl-4-yl)-2-methylpropionic acid, and pharmaceutically acceptable salts thereof and one or more steroidal anti-inflammatory agents are chosen from budesonide, pregnenolone, prednisone, prednisolone, methylprednisolone, triamcinolone, dexamethasone, betamethasone, parametasone, cortisone, and hydrocortisone. In another specific embodiment of this aspect of the invention, the dosage is provided as a pharmaceutical composition composed of (R)-2-(2-fluoro-4-biphenylyl)propionic acid and one or more steroidal anti-inflammatory agents chosen from budesonide, pregnenolone, prednisone, prednisolone, methylprednisolone, triamcinolone, dexamethasone, betamethasone, parametasone, cortisone, and hydrocortisone. In one specific embodiment, the one or more steroidal anti-inflammatory agents are chosen from budesonide, pregnenolone, prednisone, prednisolone, methylprednisolone, triamcinolone, dexamethasone, betamethasone, parametasone, cortisone, and hydrocortisone.

In a seventh aspect, the invention provides a method of selecting a regimen for treating cognitive decline in an individual desiring such treatment. The method of this aspect involves evaluating risk factors for cognitive decline. Evaluation of risk factors can include genetic testing for predisposing genes, alleles, and polymorphisms. Risk factors also refer to environmental factors like stroke, brain injury, age, and diet. Depending on the risk factor or factors associated with a particular patient a particular treatment regimen is selected for treating cognitive decline. For example, mutations in a Familial Alzheimer's disease genes such as APP, PS1 or PS2, are a risk factor. Another risk factor for cognitive decline is age. Head trauma is another risk factor for cognitive decline. Based on the patient's risk factors, a physician will prescribe a particular therapeutic treatment or prophylactic treatment suitable for the patient. In a specific embodiment of this aspect of the invention, one or more Aβ₄₂ lowering agents are chosen (R)-2-(2-fluoro-4-biphenylyl)propionic acid, 5[1-(2-Fluoro-biphenyl-4-yl)-1-methyl-ethyl]-2H-tetrazole, 2-(4-isobutyl-phenyl)-2-methyl propionic acid, 2-(2-fluoro-1,1′-biphenyl-4-yl)-2-methylpropionic acid, and pharmaceutically acceptable salts thereof, and one or more steroidal anti-inflammatory agents are chosen from budesonide, pregnenolone, prednisone, prednisolone, methylprednisolone, triamcinolone, dexamethasone, betamethasone, parametasone, cortisone, and hydrocortisone. In another specific embodiment of this aspect of the invention, the dosage is provided as a pharmaceutical composition composed of (R)-2-(2-fluoro-4-biphenylyl)propionic acid and one or more steroidal anti-inflammatory agents chosen from budesonide, pregnenolone, prednisone, prednisolone, methylprednisolone, triamcinolone, dexamethasone, betamethasone, parametasone, cortisone, and hydrocortisone. In one specific embodiment, the one or more steroidal anti-inflammatory agents are chosen from budesonide, pregnenolone, prednisone, prednisolone, methylprednisolone, triamcinolone, dexamethasone, betamethasone, parametasone, cortisone, and hydrocortisone.

In an eighth aspect, the invention provides a method of treating or preventing cancer (e.g., reducing the likelihood of getting cancer or delaying the onset of cancer). The method of this embodiment involves identifying a patient in need of treatment and administering to the patient an effective amount of one or more Aβ₄₂ lowering agents (and/or pharmaceutically acceptable salts thereof) and one or more hormonal modulating agents (and/or pharmaceutically acceptable salts thereof) in amounts sufficient to slow, stop, or reverse the progression of the cancer (or prevent/reduce the likelihood of a cancer developing). In one aspect of this embodiment, the hormonal modulating agents is chosen from steroids, non-steroids, estrogens, antiandrogens, antiestrogens, progestins, aromatase inhibitors, inhibitors of sex steroid biosynthesis, vitamin D3, vitamin D3 analogues, vitamin D3 derivatives, prolactin secretion inhibitors, and steroidal anti-inflammatory agents. In a specific embodiment of this aspect of the invention, the one or more Aβ₄₂ lowering agents are chosen (R)-2-(2-fluoro-4-biphenylyl)propionic acid, 5[1-(2-Fluoro-biphenyl-4-yl)-1-methyl-ethyl]-2H-tetrazole, 2-(4-isobutyl-phenyl)-2-methyl propionic acid, 2-(2-fluoro-1,1′-biphenyl-4-yl)-2-methylpropionic acid, and pharmaceutically acceptable salts thereof. In one specific embodiment of this aspect of the invention, the Aβ₄₂ lowering agent is (R)-2-(2-fluoro-4-biphenylyl)propionic acid or a pharmaceutically acceptable salt thereof. In one embodiment, one or hormonal modulators can be chosen from a glucocorticoid, an estrogen, or an androgen. In one embodiment, the one or more hormonal modulators can be chosen from vitamin D3, vitamin D3 analogues, and vitamin D3 derivatives. In one embodiment, the hormonal modulator is calcitriol. In another embodiment, the one or more steroids are chosen from alclometasone, amcinonide, beclomethasone, betamethasone, clobetasol, clocortolone, hydrocortisone, cortisol, cortisone, desonide, desoximetasone, dexamethasone, diflorasone, fludrocortisone, flunisolide, fluocinolone, fluocinonide, fluorometholone, flurandrenolide, halcinonide, medrysone, methylprednisolone, mometasone, paramethasone, prednisolone, prednisone, triamcinolone, and pharmaceutically acceptable salts thereof. In one embodiment of this aspect of the invention, the individual in need of treatment is one that has had radiotherapy and/or surgery for prostate cancer, and desires to reduce the risk of recurrence of prostate cancer.

Oral administration of a combination of one or more Aβ₄₂ lowering agents and one or more hormonal modulating agents for one day or more, one week or more, one month or more, two months or more, three months or more, four months or more, five months or more, six months or more, seven months or more, eight months or more, nine months or more, ten months or more, eleven months or more, twelve months or more, fourteen months or more, sixteen months or more, or eighteen months or more provides anticancer/antitumor activity, slows the progression of the cancer, and/or reduces the likelihood of having cancer.

In a specific embodiment of this aspect of the invention, a patient having prostate cancer is administered (R)-2-(2-fluoro-4-biphenylyl)propionic acid or a pharmaceutically acceptable salt thereof on a daily basis (e.g., once, twice, three time or more per day) in an amount ranging from 5 mg to 2400 mg per day and calcitriol in controlled release form on a once weekly basis.

In another specific embodiment of this aspect of the invention, a patient having a prostate cancer recurrence is administered (R)-2-(2-fluoro-4-biphenylyl)propionic acid or a pharmaceutically acceptable salt thereof on a daily basis (e.g., once, twice, three time or more per day) in an amount ranging from 5 mg to 2400 mg per day and calcitriol in controlled release form on a once weekly basis.

In an eighth aspect, the invention provides a method of treating or preventing a disease in an individual (e.g., reducing the likelihood of getting cancer or delaying the onset of cancer). The method of this embodiment involves identifying a patient in need of treatment and administering to the patient an effective amount of one or more Aβ₄₂ lowering agents (and/or pharmaceutically acceptable salts thereof) and one or more hormonal modulating agents (and/or pharmaceutically acceptable salts thereof) in a therapeutically effective amount (e.g., sufficient to slow, stop, or reverse the progression of the disease or prevent/reduce the likelihood of developing a disease). In one aspect of this embodiment, the hormonal modulating agents is chosen from steroids, non-steroids, estrogens, antiandrogens, antiestrogens, progestins, aromatase inhibitors, inhibitors of sex steroid biosynthesis, vitamin D3, vitamin D3 analogues, vitamin D3 derivatives, prolactin secretion inhibitors, and steroidal anti-inflammatory agents. In a specific embodiment of this aspect of the invention, the one or more Aβ₄₂ lowering agents are chosen (R)-2-(2-fluoro-4-biphenylyl)propionic acid, 5[1-(2-Fluoro-biphenyl-4-yl)-1-methyl-ethyl]-2H-tetrazole, 2-(4-isobutyl-phenyl)-2-methyl propionic acid, 2-(2-fluoro-1,1′-biphenyl-4-yl)-2-methylpropionic acid, and pharmaceutically acceptable salts thereof. In one specific embodiment of this aspect of the invention, the Aβ₄₂ lowering agent is (R)-2-(2-fluoro-4-biphenylyl)propionic acid or a pharmaceutically acceptable salt thereof. In one embodiment, one or hormonal modulators can be chosen from a glucocorticoid, an estrogen, or an androgen. In one embodiment, the one or more hormonal modulators can be chosen from vitamin D3, vitamin D3 analogues, and vitamin D3 derivatives. In one embodiment, the hormonal modulator is calcitriol. In another embodiment, the one or more steroids are chosen from alclometasone, amcinonide, beclomethasone, betamethasone, clobetasol, clocortolone, hydrocortisone, cortisol, cortisone, desonide, desoximetasone, dexamethasone, diflorasone, fludrocortisone, flunisolide, fluocinolone, fluocinonide, fluorometholone, flurandrenolide, halcinonide, medrysone, methylprednisolone, mometasone, paramethasone, prednisolone, prednisone, triamcinolone, and pharmaceutically acceptable salts thereof. 23As used herein, the term “(R)-2-(2-fluoro-4-biphenylyl)propionic acid” refers to the R-enantiomer of the non-steroidal anti-inflammatory drug flurbiprofen. Desirably, the formulations of the invention are substantially free of (S)-2-(2-fluoro-4-biphenylyl)propionic acid. In one aspect, at least 90% by weight (R)-2-(2-fluoro-4-biphenylyl)propionic acid to 10% by weight or less of (S)-2-(2-fluoro-4-biphenylyl)propionic acid of the total 2-(2-fluoro-4-biphenylyl)propionic acid (S+R) is in the pharmaceutical composition. In another aspect, at least 95% by weight (R)-2-(2-fluoro-4-biphenylyl)propionic acid to 5% by weight or less of (S)-2-(2-fluoro-4-biphenylyl)propionic acid of the total 2-(2-fluoro-4-biphenylyl)propionic acid (S+R) is in the pharmaceutical composition. In yet another aspect, at least 99 % by weight (R)-2-(2-fluoro-4-biphenylyl)propionic acid to 1 % by weight or less of (S)-2-(2-fluoro-4-biphenyl)propionic acid of the total 2-(2-fluoro-4-biphenylyl)propionic acid (S+R) is in the pharmaceutical composition. In yet another aspect, at least 99.9 % by weight (R)-2-(2-fluoro-4-biphenylyl)propionic acid to 0.1 % by weight or less of (S)-2-(2-fluoro-4-biphenylyl)propionic acid of the total 2-(2-fluoro-4-biphenylyl)propionic acid (S+R) is in the pharmaceutical composition.

As used herein, the term “unit dosage form” refers to a physically discrete unit, such as a capsule or tablet suitable as a unitary dosage for a human patient.

As used herein, the term “dose” or “dosage” refers the amount of active ingredient that an individual takes or is administered at one time. For example, an 800 mg (R)-2-(2-fluoro-4-biphenylyl)propionic acid dose refers to, in the case of a twice-daily dosage regimen, a situation where the individual takes 800 mg (R)-2-(2-fluoro-4-biphenylyl)propionic acid in the morning and 800 mg (R)-2-(2-fluoro-4-biphenylyl)propionic acid in the evening. The 800 mg (R)-2-(2-fluoro-4-biphenylyl)propionic acid dose can be divided into two or more dosage units, e.g., two 400 mg (R)-2-(2-fluoro-4-biphenylyl)propionic acid tablets or two 400 mg (R)-2-(2-fluoro-4-biphenylyl)propionic acid capsules. The examples describe in this definition are not intended to be limiting and are merely to illustrate various specific doses or dosages.

As used herein, “decline,” when used to characterize a disease such as Alzheimer's, or a symptom or marker thereof, means a worsening or progression of the disease, symptom or marker thereof over time from less-advanced to more-advanced. In the case of Alzheimer's disease, a decline indicates a worsening or increase in the severity of one or more behavioral, cognitive, biochemical or clinical parameters of the condition. “Decline” also indicates a progression of one or more scores on a cognition test that indicate a worsening of the condition, regardless of whether the actual, raw scores increase or not.

As used herein, “Alzheimer's disease” and “AD” are equivalent.

“A pharmaceutically acceptable prodrug” is a compound that may be converted under physiological conditions or by solvolysis to the specified compound or to a pharmaceutically acceptable salt of such compound.

“A pharmaceutically active metabolite” is intended to mean a pharmacologically active product produced through metabolism in the body of a specified compound or salt thereof. Metabolites of a compound may be identified using routine techniques known in the art and their activities determined using tests such as those described herein.

“A pharmaceutically acceptable salt” is intended to mean a salt that retains the biological effectiveness of the free acids and bases of the specified compound and that is not biologically or otherwise undesirable. A compound for use in the invention may possess a sufficiently acidic, a sufficiently basic, or both functional groups, and accordingly react with any of a number of inorganic or organic bases, and inorganic and organic acids, to form a pharmaceutically acceptable salt.

Aβ₄₂ Lowering Agents

The Aβ₄₂ lowering agents for use in the invention can be a known Aβ₄₂ lowering agents such as (R)-2-(2-fluoro-4-biphenylyl)propionic acid, 5[1-(2-Fluoro-biphenyl-4-yl)-1-methyl-ethyl]-2H-tetrazole, 2-(4-isobutyl-phenyl)-2-methyl propionic acid, or 2-(2-fluoro-1,1′-biphenyl-4-yl)-2-methylpropionic acid. Examples of Aβ₄₂ lowering agents for use in the combination formulations and treatments of the invention are given in, e.g., WO 01/78721, WO 2004/073705, WO 2004/064771, and WO 2004/074232, and PCT/US2005/009595 (each of which is herein incorporated by reference).

Aβ₄₂ lowering agents include, but are not limited to, those having the following Formulae:

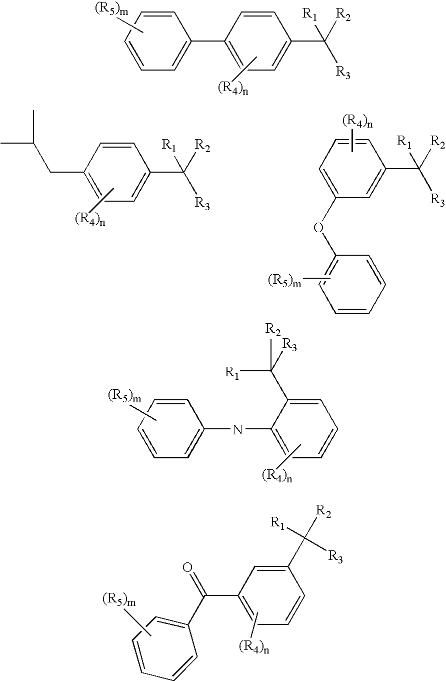

Where R₁ is chosen from —CH₃, —CH₂CH₃, —CH₂CH₂CH₃, and —CH₂CH₂CH₂CH₃ (or can be taken together with R₂ to give a cyclopropyl ring, a cyclobutyl ring, a cyclopentyl ring, or a cyclohexyl ring);

R₂ is chosen from —CH₃, —CH₂CH₃, —CH₂CH₂CH₃, and —CH₂CH₂CH₂CH₃, (or can be taken together with R₁ to give a cyclopropyl ring, a cyclobutyl ring, a cyclopentyl ring, or a cyclohexyl ring);

R₃ is chosen from —COOH, —COOR₆, —CONH₂, —CONHR₆, —CONR₆R₇, —CONHSO₂R₆, tetrazolyl, and a —COOH bioisostere;

R₄ is chosen from —Cl, —F, —Br, —I, —CF₃, —OCF₃, —SCF₃, —OCH₃, —OCH₂CH₃, —CN, —CH═CH₂, —CH₂OH, and —NO₂;

R₅ is chosen from —Cl, —F, —Br, —I, —CF₃, —OCF₃, —SCF₃, —OCH₃, —OCH₂CH₃, —CN, —CH=CH₂, —CH₂0H, and —NO₂;

R₆ is chosen from —CH₃, —CH₂CH₃, —CH₂CH₂CH₃, and —CH₂CH₂CH₂CH₃.

R₇ is chosen from —CH₃, —CH₂CH₃, —CH₂CH₂CH₃, and —CH₂CH₂CH₂CH₃.

m is an integer chosen from 0, 1, 2, and 3.

n is an integer chosen from 0, 1, 2, and 3.

Examples of compounds for use in the invention include those as shown above (and below), including enantiomers, diastereomers, racemates, and pharmaceutically acceptable salts thereof. The compounds described in this invention disclosure can be made by an ordinary artisan skilled in the art of organic chemistry synthesis.

Additional Aβ₄₂ lowering agents include: 2-methyl-2(2-fluoro-4′-trifluoromethylbiphen-4-yl) propionic acid; 2-methyl-2(2-fluoro-4′cyclohexyl biphen-4-yl) propionic acid; 1-(2-fluoro-4′-trifluoromethylbiphenyl-4-yl) cyclopropanecarboxylic acid; 1-(4′-cyclohexyl-2-fluorobiphenyl-4-yl) cyclopropanecarboxylic acid; 1-(4′-benzyloxy-2-fluorobiphenyl-4-yl) cyclopropanecarboxylic acid; 1-(2-fluoro-4′-isopropyloxybiphenyl-4-yl) cyclopropanecarboxylic acid; 1-(2-fluoro-3′-trifluoromethoxybiphenyl-4-yl) cyclopropanecarboxylic acid; 1-(2-fluoro-4′-trifluoromethoxybiphenyl-4-yl) cyclopropanecarboxylic acid; 1-(2-fluoro-3′-trifluoromethylbiphenyl-4-yl) cyclopropanecarboxylic acid; 1-(4′-cyclopentyl-2-fluorobiphenyl-4-yl) cyclopropanecarboxylic acid; 1-(4′-cycloheptyl-2-fluorobiphenyl-4-yl) cyclopropanecarboxylic acid; 1-(2′-cyclohexyl-2-fluorobiphenyl-4-yl) cyclopropanecarboxylic acid; 1-(2-fluoro-4′-hydroxybiphenyl-4-yl) cyclopropanecarboxylic acid; 1-[2-fluoro-4′- (tetrahydropyran-4-yloxy) biphenyl-4-yl]-cyclopropane-carboxylic acid; 1-(2,3′,4′-trifluorobiphenyl-4-yl) cyclopropanecarboxylic acid; 1-(3′,4′-dichloro-2-fluorobiphenyl-4-yl) cyclopropanecarboxylic acid; 1-(3′,5′-dichloro-2-fluorobiphenyl-4-yl) cyclopropanecarboxylic acid 1-(3′-chloro-2,4′-difluorobiphenyl-4-yl) cyclopropanecarboxylic acid; 1-(4-benzo [b] thiophen-3-yl-3-fluorophenyl) cyclopropanecarboxylic acid; 1-(2-fluoro-4′-prop-2-inyloxy-biphenyl-4-yl)-cyclopropanecarboxylic acid; 1-(4′-cyclohexyloxy-2-fluoro-biphenyl-4-yl)-cyclopropanecarboxylic acid; 1-[2-fluoro-4′- (tetrahydropyran-4-yl)-biphenyl-4-yl]-cyclopropanecarboxylic acid; 1-[2-fluoro-4′-(4-oxo-cyclohexyl)-biphenyl-4-yl]-cyclopropanecarboxylic acid; 2-(2″-fluoro-4-hydroxy-[1,1′:4′,1″]tert-phenyl-4″-yl)-cyclopropanecarboxylic acid; 1-[4′-(4,4-dimethylcyclohexyl)-2-fluoro[1,1′-biphenyl]-4-yl]-cyclopropane-carboxylic acid; 1-[2-fluoro-4′-[[4-(trifluoromethyl) benzoyl]amino][1,1′-biphenyl]-4-yl]-cyclopropanecarboxylic acid; 1-[2-fluoro-4′-[[4-(trifluoromethyl)cyclohexyl]oxy][1,1′-biphenyl]-4-yl]-cyclopropanecarboxylic acid; 1-[2-fluoro-4′-[(3,3,5,5-tetramethylcyclohexyl)oxy][1,1′-biphenyl]-4-yl]-cyclopropanecarboxylic acid; 1-[4′-[(4,4-dimethylcyclohexyl)oxy]-2-fluoro[1,1′-biphenyl]-4-yl]-cyclopropanecarboxylic acid; 1-(2,3′,4″-trifluoro[1,1′:4′,1″-tert-phenyl]-4-yl)-cyclopropanecarboxylic acid; 1-(2,2′,4″-trifluoro[1,1′:4′,1″-tert-phenyl]-4-yl)-cyclopropanecarboxylic acid; 1-(2,3′-difluoro-4″-hydroxy[1,1′:4′,1″-tert-phenyl]-4-yl)-cyclopropane-carboxylic acid; 1-(2,2′-difluoro-4″-hydroxy [1,1′:4′,1″-tert-phenyl]-4-yl)-cyclopropane-carboxylic acid; 2-(2-fluoro-3′,5′-bis (chloro) biphen-4-yl) propionic acid amide; 2-(2-fluoro-4′-trifluoromethylbiphen-4-yl) propionic acid; 2-(2-fluoro-3′-trifluoromethylbiphen-4-yl) propionic acid; 2-(2-fluoro-3′,5′-bis(trifluoromethyl)biphen-4-yl)propionic acid; 2-(4′-cyclohexyl-2-fluorobiphen-4-yl)propionic acid; 2-(2-Fluoro-1,1′-biphenyl-4-yl)-2-methylpropanoic acid; 2-Methyl-2-(3-phenoxy-phenyl)-propionic acid; 2-(4-Isobutyl-phenyl)-2-methyl-propionic acid; 2-(6-Chloro-9H-carbazol-2-yl)-2-methyl-propionic acid; 2-[1-(4-Chloro-benzoyl)-5-methoxy-2-methyl-1H-indol-3-yl]-2-methyl-propionic acid; and 5-[1-(2-Fluoro-biphenyl-4-yl)-1-methyl-ethyl]-2H-tetrazole.

One specific Aβ₄₂ lowering agent for use in the invention is (R)-2-(2-fluoro-4-biphenylyl)propionic acid. Methods of resolving (R)-2-(2-fluoro-4-biphenylyl)propionic acid from the racemate are disclosed in U.S. Pat. No. 5,599,969 to Hardy et al. which discloses contacting the racemates with α-methylbenzylamine salt in a solvent mixture of toluene and methanol, followed by recrystallization of the diastereomer salt. The diastereomer salts are then separated to give the resolved flurbiprofen enantiomers. U.S. Pat. No. 4,209,638 to Boots Co. discloses a process for resolving 2-arylproprionic acids which include flurbiprofen by mixing the racemate with a chiral organic nitrogenous base under certain conditions followed by recovery and separation of the diastereomeric salts. Other patents disclosing processes for resolving racemic arylproprionic acids include U.S. Pat. No. 4,983,765 to PAZ; U.S. Pat. No. 5,015,764 to Ethyl Corp.; U.S. Pat. No. 5,235,100 to Ethyl Corp.; U.S. Pat. No. 5,574,183 to Albemarle Corp.; U.S. Pat. No. 5,510,519 to Sumitomo Chemical Company. Methods of tableting (R)-2-(2-fluoro-4-biphenylyl)propionic acid and arylproprionic acids are disclosed in, e.g., U.S. Pat. No. 5,667,807 to Hurner et al.; U.S. Pat. No. 5,565,613 to Geisslinger at al.; U.S. Pat. No. 6,471,991 to Robinson et al.; and U.S. Pat. No. 6,379,707 to Vladyka et al.

Aβ₄₂ lowering agents can be identified by a number of methods. To identify Aβ₄₂ lowering agents that reduce APP processing, a biological composition having an APP processing activity (i.e. an activity that processes APP into various Aβ forms, one of which is Aβ₄₂), is incubated with APP under conditions in which APP processing occurs. To identify Aβ₄₂ lowering agents that increase Aβ₄₂ catabolism, a biological composition having Aβ₄₂ catabolic activity is incubated with Aβ₄₂ under conditions in which Aβ₄₂ catabolism occurs. Depending on the nature of the biological composition, the APP or Aβ₄₂ substrate can be added to the biological composition, or, each or both can be a component of the biological composition. APP processing or Aβ₄₂ catabolism is allowed to take place in the presence or absence of the candidate Aβ₄₂ lowering agent. The level of Aβ₄₂ generated from APP processing or the level of Aβ₄₂ remaining after the catabolic reaction, in the presence and absence of the candidate Aβ₄₂ lowering agent, is determined and compared. Aβ₄₂ lowering agents useful for treating AD are those that reduce the level of Aβ₄₂ either by reducing APP processing into Aβ₄₂ or by enhancing Aβ₄₂ catabolism and increasing Aβ₃₈ production. The biological composition having an APP processing and/or catabolic activity can be a cell-free biological sample. For example, a cell-free biological sample can be a purified or partially purified enzyme preparation; it also can be a cell lysate generated from cells able to process APP into Aβ₄₂ or from cells able to catabolize Aβ₄₂. Cell lysates can be prepared using known methods such as, for example, sonication or detergent-based lysis. In the case of an enzyme preparation or cell lysate, APP can be added to the biological composition having the APP processing activity, or Aβ₄₂ can be added to the biological composition having Aβ₄₂ catabolic activity.

In addition, the biological composition can be any mammalian cell that has an APP processing activity as well as a nucleic acid vector encoding APP. Alternatively, the biological composition can be any mammalian cell that has Aβ catabolic activity as well as a nucleic acid vector or a viral nucleic acid-based vector containing a gene that encodes Aβ₄₂. The vector typically is an autonomously replicating molecule, a molecule that does not replicate but is transiently transfected into the mammalian cell, or a vector that is integrated into the genome of the cell. Typically, the mammalian cell is any cell that can be used for heterologous expression of the vector-encoded APP or Aβ₄₂ in tissue culture. For example, the mammalian cell can be a Chinese hamster ovary (CHO) cell, a fibroblast cell, or a human neuroglioma cell. The mammalian cell also can be one that naturally produces APP and processes it into Aβ₄₂, or one that naturally produces and catabolizes Aβ₄₂.

Further, the biological composition can be an animal such as a transgenic mouse that is engineered to over-express a form of APP that then is processed into Aβ₄₂. Alternatively, the animal can be a transgenic mouse that is engineered to over-express Aβ₄₂. Animals can be, for example, rodents such as mice, rats, hamsters, and gerbils. Animals also can be rabbits, dogs, cats, pigs, and non-human primates, for example, monkeys.

To perform an in vitro cell-free assay, a cell-free biological sample having an activity that can process APP into Aβ₄₂ is incubated with the substrate APP under conditions in which APP is processed into various Aβ forms including Aβ₄₂ (see Mclendon et al. (2000) FASEB 14:2383-2386). Alternatively, a cell-free biological sample having an activity that can catabolize Aβ₄₂ is incubated with the substrate Aβ₄₂ under conditions in which Aβ₄₂ is catabolized. To determine whether a candidate Aβ₄₂ lowering agent has an effect on the processing of APP into Aβ₄₂ or the catabolism of Aβ₄₂, two reactions are compared. In one reaction, the candidate Aβ₄₂ lowering agent is included in the processing or catabolic reaction, while in a second reaction, the candidate Aβ₄₂ lowering agent is not included in the processing or catabolic reaction. Levels of the different Aβ forms produced in the reaction containing the candidate Aβ₄₂ lowering agent are compared with levels of the different AP forms produced in the reaction that does not contain the candidate Aβ₄₂ lowering agent.

The different AP forms can be detected using any standard antibody based assays such as, for example, immunoprecipitation, western hybridization, and sandwich enzyme-linked immunosorbent assays (ELISA). Different Aβ forms also can be detected by mass spectrometry; see, for example, Wang et al. (1996) J Biol Chem 271:31894-902. Levels of Aβ species can be quantified using known methods. For example, internal standards can be used as well as calibration curves generated by performing the assay with known amounts of standards.

In vitro cell-based assays can be used determine whether a candidate Aβ₄₂ lowering agent has an effect on the processing of APP into Aβ₄₂ or an effect on catabolism of Aβ₄₂. Typically, cell cultures are treated with a candidate Aβ₄₂ lowering agent. Then the level of Aβ₄₂ in cultures treated with a candidate Aβ₄₂ lowering agent is compared with the level of Aβ₄₂ in untreated cultures. For example, mammalian cells expressing APP are incubated under conditions that allow for APP expression and processing as well as Aβ₄₂ secretion into the cell supernatant. The level of Aβ₄₂ in this culture is compared with the level of Aβ₄₂ in a similarly incubated culture that has been treated with the candidate Aβ₄₂ lowering agent. Alternatively, mammalian cells expressing Aβ₄₂ are incubated under conditions that allow for Aβ₄₂ catabolism. The level of Aβ₄₂ in this culture is compared with the level of Aβ₄₂ in a similar culture that has been treated with the candidate Aβ₄₂ lowering agent.

In vivo animal studies also can be used to identify Aβ₄₂ lowering agents useful for treating AD. Typically, animals are treated with a candidate Aβ₄₂ lowering agent and the levels of Aβ₄₂ in plasma, CSF, and/or brain are compared between treated animals and those untreated. The candidate Aβ₄₂ lowering agent can be administered to animals in various ways. For example, the candidate Aβ₄₂ lowering agent can be dissolved in a suitable vehicle and administered directly using a medicine dropper or by injection. The candidate Aβ₄₂ lowering agent also can be administered as a component of drinking water or feed. Levels of Aβ in plasma, cerebral spinal fluid (CSF), and brain are determined using known methods. For example, levels of Aβ₄₂ can be determined using sandwich ELISA or mass spectrometry in combination with internal standards or a calibration curve. Plasma and CSF can be obtained from an animal using standard methods. For example, plasma can be obtained from blood by centrifugation, CSF can be isolated using standard methods, and brain tissue can be obtained from sacrificed animals.

When present in an in vitro or in vivo APP processing or Aβ₄₂ catabolic reaction, Aβ₄₂ lowering agents reduce the level of Aβ₄₂ generated by APP processing or remaining following Aβ catabolism. For example, in an in vitro cell-free assay, the level of Aβ₄₂ is reduced due to either a reduction of APP processing or an increase in Aβ₄₂ catabolism in the presence the Aβ₄₂ lowering agent. In an in vitro cell culture study, a reduction in the level of Aβ₄₂ secreted into the supernatant results from the effect of the Aβ₄₂ lowering agent on either a reduction in processing of APP into Aβ₄₂ or an increased catabolism of Aβ₄₂. Similarly, in animal studies, a reduction in the level of Aβ₄₂ that can be detected in plasma, CSF, or brain is attributed to the effect of the Aβ₄₂ lowering agent on either a reduction in the processing of APP into Aβ₄₂ or an increase in the catabolism of Aβ₄₂.

The level of Aβ₄₂ can be reduced by a detectable amount. For example, treatment with an Aβ₄₂ lowering agent leads to a 0.5, 1, 3, 5, 7, 15, 20, 40, 50, or more than 50% reduction in the level of Aβ₄₂ generated by APP processing or remaining following Aβ₄₂ catabolism when compared with that in the absence of the Aβ₄₂ lowering agent. In one specific embodiment, treatment with the Aβ₄₂ lowering agent leads to a 20% or more reduction in the level of Aβ₄₂ generated when compared to that in the absence of Aβ₄₂ lowering agent. In another specific embodiment, treatment with an Aβ₄₂ lowering agent leads to a 40% or more reduction the level of Aβ₄₂ when compared to that in the absence of an Aβ₄₂ lowering agent,

Hormonal Modulator

The combination treatments of the invention include one or more hormonal modulators. According to one embodiment, the hormonal modulators can be chosen from steroids, non-steroids, estrogens, antiandrogens, antiestrogens, progestins, aromatase inhibitors, inhibitors of sex steroid biosynthesis, Vitamin D3 and its derivatives, prolactin secretion inhibitors, and steroidal anti-inflammatory agents.

Antiandrogens

Antiandrogens block the body's ability to use any androgens. In one embodiment, one or more compounds are chosen from flutamide (Eulexin), bicalutamide (Casodex), and nilutamide (Nilandron).

Antiestrogens

The antiestrogen compounds useful in the various aspects of this invention include many compounds that are well known in the art. In some embodiments, the antiestrogen is chosen from tamoxifen ((Z)-2-[4-(1,2-diphenyl-1-butenyl)-phen-oxyl]-N,N-dimethylethanamine or 1-p-β-dimethylaminoethoxyphenyl-trans- -1,2-diphenylbut-1-ene) and toremifene ((Z)-2-[4-(4-chloro-1,2-diphenyl-1-but-enyl)phenoxy]-N,N-dimethylethanamine), along with the pharmaceutically acceptable salts of these compound. Commercially available as Novaldex® from Astra Zeneca Pharmaceuticals and Fareston® from Shire Pharmaceuticals. Others are set forth in U.S. Pat. No. 5,550,107 to Labrie, which is incorporated herein by reference.

In some embodiments, the antiestrogen is chosen from steroidal and non-steroidal antiestrogens. In some embodiments, the antiestrogen is chosen from (1RS,2RS)-4,4′-diacetoxy-5,5′-difluoro-(1-ethyl-2-methylene)di-m-phenylen-ediacetate (from Biorex); 6α-chloro-16α-methyl-pregn-4-ene-3,20-dione (from Eli Lilly & Co., Indianapolis, Ind.); 6-chloro-17-hydroxypregna-1,4,6-triene-3,20-dione (Delmadione Acetate); 17-hydroxy-6-methyl-19-norpregna-4,6-diene-3,20-dione (from Theramex); 1-[2-[4-[1-4(4-methoxyphenyl)-2-nitro-2-phenylethenyl)phenoxy]ethyl]-pyrrolidin (Nitromifene); substituted aminoalkoxyphenylalkenes (e.g., tamoxifen citrate salt from Stuart Pharmaceuticals, Wilmington, Del. (see also Belgian patent No. 637,389, March 1964)); 3,4-dihydro-2-(p-methoxyphenyl)-1-naphthyl p-[2-(1-pyrrolidinyl)ethoxy]phenyl ketone (available as the methane sulfonate salt from Eli Lilly & Co); 1-[4′-(2-phenyl)-bl-(3′-hydroxyphenyl)-2-phenyl-but-1-ene (available from Klinge Pharma); [6-hydroxy-2-(p-hydroxyphenyl-)-benzo(b)thien-3yl]-[2-(1-pyrrolidinyl)-ethoxy phenyl]ketone ((LY 117018) available from Eli Lilly & Co.); [6-hydroxy-2-(4-hydroxyphenyl)benzo(b)thien-3-yl]-[4-(2-(1-piperdinyl)ethoxy)phenyl]methanone ((LY 156758) available from Eli Lilly & Co.); meso-3,4-bis(3′-hydroxyphenyl) hexane as well as the dimethyl, dipropyl and 3′-acetoxy phenyl analogues which are described in U.S. Pat. No. 4,094,994; and a series of 1-phenyl-alkane and -alkenes, e.g., (E)-3-cyclopentyl-1-(4-hydroxyphenyl)-1-phenyl-1-butene and 2-cyclo-pentyl-1-[4-hydroxy or methoxyphenyl]-3-phenyl-2-propen-1-ol and FC-1157 which are available as the citrate salt from Farmos Group, Ltd., Turku, Finland (see also Eur. Pat. Appln. Ep. No 78,158).

In some embodiments, one or more antiestrogens are chosen from 7α-substituents of estradiol (European Pat. No. 0138504) and non-steroidal compounds bearing a similar aliphalic side-chain (U.S. Pat No. 4,732,912), both of which are incorporated herein by reference.

In some embodiments, one or more compounds are chosen from 4-methyl-2[4-[2-(1-piperidinyl)ethoxy]phenyl]-7-(pivaloyloxy)-3-[4-(pivaloyloxy)phenyl]-2H-1-benzopyran (code name EM-800); lasofoxifene (5,6,7,8-tetrahydro-6-phenyl-5-(4-(2-(1-pyrrolidinyl)ethoxy)phenyl-(5R-cis)-2-naphthalenol, (S-(R*, R*))-2,3-dihydroxybutanedioate) by Ligand; 2-methoxyestradiol by EntreMed; LY-326391 ((2-(4-methoxyphenyl)-3-(4-(2-(1-piperidinyl)ethoxy)phenoxy)-benzo(b)thiophene-6-ol, hydrochloride) by Eli Lilly; A-007 (4,4′-dyhydroxybenzophenone-2,4-dinitrophenylhydrazone) by Dekk-Tec; fluvestront ((7α, 17β)-7-[9-[(4,4,5,5,5-pentafluoropentyl)sulfinyl]nonyl]-estra-1,3,5(10)-triene-3,17-diol) by AstraZeneca.

In one embodiment, the combinations and co-formulations include one or more Aβ₄₂ lowering agents and one or more inhibitors of sex steroid biosynthesis. In one aspect of this embodiment, inhibitors of sex steroid biosynthesis can be chosen from aminoglutethimide, ketoconazole, 4-hydroxyandrostenedione, atamestane, exemestane, anastrazole, fadrozole, finrozole, letrozole, vorozole, and YM-511 In one aspect of this embodiment, the inhibitors of sex steroid biosynthesis include 3-(4-aminophenyl)-3-ethyl-2,6-piperidinedione (commonly know as aminoglutethimide, available from Ciba Pharmaceutical Co., Summit N.J.), and ketoconazole (available from Janssen Pharmaceuticals, Piscataway, N.J.). Another inhibitor is 4-hydroxyandrostenedione. In one aspect of this embodiment, the inhibitors of sex steroid biosynthesis are chosen from atamestane (1-methylandrosta-1,4-diene-3,17-dione); exemestane (6-Methylene androsta-1,4-diene-3,17-dione); anastrazole (α, α, α, α′-tetramethyl-5-(1H-1,2,4-triazol-1-ylmethyl)-1,3-benzenediacetonitrile); fadrozole (4-(5,6,7,8-Tetrahydroimidazo[1,5 -a]pyridin-5-yl)-benzonitrile, monohydrochloride); finrozole (4-(3-(4-Fluorophenyl)-2-hydroxy-1-(1H-1,2,4-triazol-1-yl-)-propyl)-benzonitrile); letrozole (4,4′-(1H-1,2,4-triazol-1-ylmethylene)bis-benzonitrile); vorozole (6-[(4-chlorophenyl)-1H- 1,2,4-triazol-1-ylmethyl]-1-methyl-1H-benzotriazole); and YM-511 (4-[N-(4-bromobenzyl)-N-(4-cyanophenyl)amino]-4H-1,2,4-triazole).

Inhibitors of 3β-hydroxysteroid or Δ5-Δ4-isomerase activity, such as Trilostane, Eposlane or 4-MA, are also useful. Others, such as 16-methylene estrone and 16-methylene estradiol, act as specific inhibitors of 17β-estradiol dehydrogenase (Thomas et al. J. Biol. Chem. 258: 11500-11504, 1983).

Progestins

In some embodiments, one or more compounds can be progestins. In one embodiment, the combination is used for treating ER-negative, PR-positive cancers and hormone-independent breast cancer following PR reactivation (ER: estrogen, PR: progesterone receptor).

A progestin is a substance that affects some or all of the changes produced by progesterone. In some embodiment, the progestin is chosen from 17,21-dimethyl-19-nor-4,9-pregnadiene-3,20-dione (available from Roussel-UCLAF); cyproterone acetate (available from Schering Ag.); 6-α-methyl, 17-α-acetoxy progesterone or medroxyprogesterone acetate (available from Upjohn and Farmitalia, Calbo Erba); Gestoden (available from Shering); magestrol acetate (17α-acetoxy-6-methyl-pregna-4,6-diene-3,20-dione) (available from Mead Johnson & Co., Evansville, Ind). In some embodiments, the progestin is chosen from levolorgestrel, gestodene, desogestrel, 3-keto-desogestrel, norethindrone, norethisterone, 13α-ethyl-17-hydroxy-18,19-dinor- 17β-pregna-4,9,11-triene-20-y-1-3-one (R2323), demegestone, norgestrienone, gastrinone, progesterone itself, and others described in Raynaud et al. J. Steroid Biochem. 25: 811-833, 1986; Raynaud et al., J. Steroid Biochem. 12: 143-157, 1980; Raynaud et al. Steroid Hormones, Agonists and Antagonists, In: Mechanisms of Steroid Action (G. P. Lewis and M. Ginsburg, eds), McMillan Press, London, pp. 145-158 (1981).

Prolactin Secretion Inhibitors

In some embodiments, one or more compounds are chosen from prolactin secretion inhibitors, which are agents that reduce the production of the protein hormone prolactin from the pituitary gland. In one embodiment, the prolactin secretion inhibitor is bromocriptine (available from Novartis).

Vitamin D3 Related Compounds

In one embodiment, the one or more hormonal modulators can be chosen from calciol (or cholecalciferol), ercalciol (or ergocalciferol), calcidiol, (1S)-hydroxycalciol, (24R)-hydroxycalcidiol, calcitriol, calcitetrol, 25-fluorocalciol, ercalcidiol, ercalcitriol, ertacalciol, tacalciol, (5E)-isocalciol, 22,23-dihydroercalciol (or (24S)-methylcalciol), (5E)-(10S)-10,19-dihydroercalciol, (6Z)-tacalciol, (24S)-ethylcalciol, (22E)-(24R)-ethyl-22,23-didehydrocalciol, 25-Dihydroxy-20epi-22-oxa-24,26,27-trisho-mocholecalciferol (KH 1060), 1,25-Dihydroxy-22E,24E-diene-24,26,27-trishomocholecalciferol (EB 1039), and 1,25-Dihydroxy-16-ene-24-oxo-19-norcholecalciferol.

Steroidal Anti-Inflammatory Agents

In some embodiments, one or more steroidal anti-inflammatory agents are chosen from mineralosteroids and glucorticosteroids. In some embodiments, one or more steroidal anti-inflammatory agents are chosen from those having a pregna-1,4-diene-3,20 dione core structure. In one aspect of this embodiment, the one or more steroidal anti-inflammatory agents having a pregna-1,4-diene-3,20 dione core structure are chosen from dexamethasone, fluorometholone, betamethasone, corticosterone, and prednisolone.

Examples of steroidal anti-inflammatory agents include, but are not limited to, budesonide, pregnenolone, prednisone, prednisolone, methylprednisolone, triamcinolone, dexamethasone, betamethasone, parametasone, cortisone, and hydrocortisone.

In some aspects of the invention dexamethasone is used in the combination treatments and co-formulations. Dexamethasone is available from a number of sources including Par Pharmaceuticals (Woodcliff Lake, N.J.). In one embodiment, dexamethasone is administered in an amount from about 0.25 mg to about 10 mg per day. In one aspect of this embodiment, dexamethasone is administered in an amount from about 0.25 mg to about 7.5 mg, 0.25 mg to about 6.5 mg, 0.25 mg to about 5.5 mg, 0.25 mg to about 5.0 mg, or 0.25 mg to about 4.5 mg per day. In one aspect of this embodiment, dexamethasone is administered in an amount from about 10 or less mg, 9 or less mg, 8 or less mg, 7 or less mg, 6 or less mg, or 5 or less mg per day. In one aspect of this embodiment, dexamethasone is administered in an amount from about 0.25 or more mg, 1 or more mg, 2 or more mg, 3 or more mg, 4 or more mg, or 5 or more mg per day.

In some aspects of the invention budesonide is used in the combination treatments and co-formulations. Budesonide is available from a number of sources including Astrazeneca (Willimgtom, Del.). In one embodiment, budesonide is administered in an amount from about 0.25 mg to about 10 mg per day. In one aspect of this embodiment, budesonide is administered in an amount from about 0.25 mg to about 7.5 mg, 0.25 mg to about 6.5 mg, 0.25 mg to about 5.5 mg, 0.25 mg to about 5.0 mg, or 0.25 mg to about 4.5 mg per day. In one aspect of this embodiment, budesonide is administered in an amount from about 10 or less mg, 9 or less mg, 8 or less mg, 7 or less mg, 6 or less mg, or 5 or less mg per day. In one aspect of this embodiment, cortisone is administered in an amount from about 0.25 or more mg, 1 or more mg, 2 or more mg, 3 or more mg, 4 or more mg, or 5 or more mg per day.

In some aspects of the invention cortisone is used in the combination treatments and co-formulations. Cortisone is available from a number of sources including Pfizer (NY, N.Y.). In one embodiment, cortisone is administered in an amount from about 10 mg to about 300 mg per day. In one aspect of this embodiment, cortisone is administered in an amount from about 10 mg to about 275 mg, 10 mg to about 250 mg, 10 mg to about 200 mg, 10 mg to about 175 mg, or 10 mg to about 150 mg per day. In one aspect of this embodiment, cortisone is administered in an amount from about 200 or less mg, 150 or less mg, 125 or less mg, 100 or less mg, 90 or less mg, or 80 or less mg per day. In one aspect of this embodiment, cortisone is administered in an amount from about 20 or more mg, 30 or more mg, 40 or more mg, 50 or more mg, 60 or more mg, or 70 or more mg per day.

In some aspects of the invention betamethasone is used in the combination treatments and co-formulations. Betamethasone is available from a number of sources including Schering-Plough (Kenilworth, N.J.). In one embodiment, betamethasone is administered in an amount from about 0.25 mg to about 7.5 mg per day. In one aspect of this embodiment, betamethasone is administered in an amount from about 0.25 to about 6.5 mg, 0.25 mg to about 5.5 mg, 0.25 mg to about 5.0 mg, or 0.25 mg to about 4.5 mg per day. In one aspect of this embodiment, betamethasone is administered in an amount from about 7.5 or less mg, 7 or less mg, 6.5 or less mg, 6 or less mg, 5.5 or less mg, or 5 or less mg per day. In one aspect of this embodiment, betamethasone is administered in an amount from about 0.25 or more mg, 1 or more mg, 2 or more mg, 3 or more mg, 4 or more mg, or 5 or more mg per day.

In some aspects of the invention hydrocortisone is used in the combination treatments and co-formulations. Hydrocortisone is available from a number of sources including Pfizer (NY, N.Y.). In one embodiment, hydrocortisone is administered in an amount from about 20 mg to about 800 mg per day. In one aspect of this embodiment, hydrocortisone is administered in an amount from about 20 mg to about 700 mg, 20 mg to about 600 mg, 20 mg to about 500 mg, 20 mg to about 400 mg, or 20 mg to about 300 mg per day. In one aspect of this embodiment, hydrocortisone is administered in an amount from about 700 or less mg, 600 or less mg, 500 or less mg, 400 or less mg, 300 or less mg, or 200 or less mg day. In one aspect of this embodiment, hydrocortisone is administered in an amount from about 20 or more mg, 30 or more mg, 40 or more mg, 50 or more mg, 60 or more mg, or 70 or more mg day.

In some aspects of the invention methylprednisolone is used in the combination treatments and co-formulations. Methylpredisolone is available from a number of sources including Par Pharmaceutical (Woodcliff Lake, N.J.). In one embodiment, methylprednisolone is administered in an amount from about 4 mg to about 160 mg per day. In one aspect of this embodiment, methylprednisolone is administered in an amount from about 4 mg to about 140 mg, 4 mg to about 120 mg, 4 mg to about 100 mg, or 4 mg to about 80 mg per day. In one aspect of this embodiment, methylprednisolone is administered in an amount from about 150 or less mg, 140 or less mg, 130 or less mg, 120 or less mg, 110 or less mg, or 100 or less mg per day. In one aspect of this embodiment, methylprednisolone is administered in an amount from about 4 or more mg, 10 or more mg, 20 or more mg, 30 or more mg, 40 or more mg, or 50 or more mg per day.

In some aspects of the invention prednisolone is used in the combination treatments and co-formulations. Prednisolone is available from a number of sources including Par Pharmaceuticals (Woodcliff Lake, N.J.). In one embodiment, prednisolone is administered in an amount from about 5 mg to about 200 mg per day. In one aspect of this embodiment, prednisolone is administered 5 mg to about 180 mg, 5 mg to about 150 mg, 5 mg to about 125 mg, or 5 mg to about 100 mg per day. In one aspect of this embodiment, prednisolone is administered in an amount from about 200 or less mg, 150 or less mg, 125 or less mg, 100 or less mg, 90 or less mg, or 80 or less mg per day. In one aspect of this embodiment, prednisolone is administered in an amount from about 5 or more mg, 10 or more mg, 20 or more mg, 30 or more mg, 40 or more mg, or 50 or more mg per day.

In some aspects of the invention prednisone is used in the combination treatments and co-formulations. Prednisone is available from a number of sources including Watson Pharmaceuticals (Coronona, Calif.). In one embodiment, prednisone is administered in an amount from about 5 mg to about 200 mg per day. In one aspect of this embodiment, prednisone is administered 5 mg to about 180 mg, 4 mg to about 150 mg, 5 mg to about 125 mg, or 4 mg to about 100 mg per day. In one aspect of this embodiment, prednisone is administered in an amount from about 200 or less mg, 150 or less mg, 125 or less mg, 100 or less mg, 90 or less mg, or 80 or less mg per day. In one aspect of this embodiment, prednisone is administered in an amount from about 20 or more mg, 30 or more mg, 40 or more mg, 50 or more mg, 60 or more mg, or 70 or more mg per day.

In some aspects of the invention triamcinolone is used in the combination treatments and co-formulations. Triamcinolone is available from a number of sources including Astellas Pharma (Deerfield, Ill.). In one embodiment, triamcinolone is administered in an amount from about 2 mg to about 60 mg per day. In one aspect of this embodiment, triamcinolone is administered 2 mg to about 50 mg, 2 mg to about 45 mg, 2 mg to about 35 mg, or 2 mg to about 30 mg per day. In one aspect of this embodiment, triamcinolone is administered in an amount from about 60 or less mg, 50 or less mg, 40 or less mg, 35 or less mg, 30 or less mg, or 25 or less mg per day. In one aspect of this embodiment, triamcinolone is administered in an amount from about 2 or more mg, 5 or more mg, 10 or more mg, 15 or more mg, 20 or more mg, or 25 or more mg day per day.

Other Steroidal Agents

In some aspects of the invention pregnenolone is used in the combination treatments and co-formulations. Pregnenolone is available from a number of sources and has a CAS number of [145-13-1]. In one embodiment, pregnenolone is administered in an amount from about 5 mg to about 1000 mg per day. In one aspect of this embodiment, pregnenolone is administered 5 mg to about 500 mg, 5 mg to about 400 mg, 5 mg to about 300 mg, or 5 mg to about 200 mg per day. In one aspect of this embodiment, pregnenolone is administered in an amount from about 200 or less mg, 150 or less mg, 125 or less mg, 100 or less mg, 90 or less mg, or 80 or less mg per day. In one aspect of this embodiment, pregnenolone is administered in an amount from about 20 or more mg, 30 or more mg, 40 or more mg, 50 or more mg, 75 or more mg, or 100 or more mg per day.

In some aspects of the invention DHEA (Dehydroepiandrosterone) is used in the combination treatments and co-formulations. DHEA is available from a number of sources and has a CAS number of [53-43-0]. In one embodiment, pregnenolone is administered in an amount from about 5 mg to about 300 mg per day. In one aspect of this embodiment, DHEA is administered 5 mg to about 250 mg, 5 mg to about 200 mg, 5 mg to about 150 mg, or 5 mg to about 100 mg per day. In one aspect of this embodiment, DHEA is administered in an amount from about 200 or less mg, 150 or less mg, 125 or less mg, 100 or less mg, 90 or less mg, or 80 or less mg per day. In one aspect of this embodiment, prednisone is administered in an amount from about 20 or more mg, 30 or more mg, 40 or more mg, 50 or more mg, 75 or more mg, or 100 or more mg per day.

In some aspects of the invention 7β-hydroxy epiandrosterone is used in the combination treatments and co-formulations. 7β-hydroxy epiandrosterone is available from a number of sources. In one embodiment 7β-hydroxy epiandrosterone is administered in an amount from about 5 mg to about 1000 mg per day. In one aspect of this embodiment, 7β-hydroxy epiandrosterone is administered 5 mg to about 500 mg, 5 mg to about 400 mg, 5 mg to about 300 mg, or 5 mg to about 200 mg per day. In one aspect of this embodiment, 7β-hydroxy epiandrosterone is administered in an amount from about 200 or less mg, 150 or less mg, 125 or less mg, 100 or less mg, 90 or less mg, or 80 or less mg per day. In one aspect of this embodiment, 7β-hydroxy epiandrosterone is administered in an amount from about 20 or more mg, 30 or more mg, 40 or more mg, 50 or more mg, 75 or more mg, or 100 or more mg per day.

Patient Population

Any individual having, or suspected of having, a neurodegenerative disorder, such as Alzheimer's disease, may be treated using the compositions and methods of the present invention. Individuals who would particularly benefit from the compositions and methods of the invention include those individuals diagnosed as having mild to moderate Alzheimer's disease according to a medically-accepted diagnosis, such as, for example the NINCDS-ADRDA criteria. Progression of the disease may be followed by medically accepted measure of cognitive function, such as, for example, the Mini-Mental State Exam (MMSE; see Mohs et al. Int. Psychogeriatr. 8:195-203 (1996)); ADAS-Cog (Alzheimer Disease Assessment Scale-Cognitive; see Galasko et al. Alzheimer Dis Assoc Disord, 11 suppl 2:S33-9 (1997)); Behavioral Pathology in Alzheimer's Disease Rating Scale (BEHAVE-AD); Blessed Test; CANTAB - Cambridge Neuropsychological Test Automated Battery; CERAD (The Consortium to Establish a Registry for Alzheimer's Disease) Clinical and Neuropsychological Tests (includes MMSE); Clock Draw Test; Cornell Scale for Depression in Dementia (CSDD); Geriatric Depression Scale (GDS); Neuropsychiatric Inventory (NPI); the 7 Minute Screen; the Alzheimer's Disease Cooperative Study Activities of Daily Living scale (ADCS-ADL; see McKhann et al. Neurology 34:939-944 (1984)); the DSM-IV (Diagnostic and Statistical Manual of Mental Disorders—Fourth Edition (DSM-IV), published by the American Psychiatric Association, Washington D.C., 1994); or the NINCDS-ADRDA criteria (see Folstein et al. J. Psychiatr. Res. 12:189-198 (1975)). Individuals diagnosed as having probable AD can be identified as having a mild-to-moderate form of the disease by an accepted measure of cognitive function such as the MMSE. In addition, methods that allow for evaluating different regions of the brain and estimating plaque and tangle frequencies can be used. These methods are described by Braak et al. Acta Neuropathol 82:239-259 (1991); Khachaturian Arch. Neuro. 42:1097-1105 (1985); Mirra et al. (1991) Neurology 41:479-486; and Mirra et al. Arch Pathol Lab Med 117:132-144 (1993). The severity of AD is generally determined by one of the initial tests provided above. For example, MMSE scores of 26-19 indicate mild AD, while scores from 18-10 indicate moderate AD.

Diagnoses of Alzheimer's disease based on these tests are recorded as presumptive or probable, and may optionally be supported by one or more additional criteria. For example, a diagnosis of Alzheimer's disease may be supported by evidence of a family history of AD; non-specific changes in EEG, such as increased slow-wave activity; evidence of cerebral atrophy on CT with progression documented by serial observation; associated symptoms such as depression, insomnia, incontinence, delusions, illusions, hallucinations, catastrophic verbal, emotional or physical outbursts, sexual disorders, weight loss, and/or attendant neurologic abnormalities, such as increased muscle tone, myoclonus or gait disorder, etc.

Additionally, amyloid deposits, generally associated with AD, may be detected through the use of positron emission tomography (PET) using an amyloid -specific tracer such as Pittsburgh Compound-B (PIB). See Klunk et al., Ann. Neurol. 55(3):306-309 (2004). Increased amyloid deposits in the frontal, parietal, temporal and occipital cortices, and in the striatum, relative to normal brain tissue, as visualized, for example by PIB, support a diagnosis of AD. Generally, a greater number and density of amyloid deposits indicates more advanced AD.

The invention encompasses the treatment of an individual preferably having mild to moderate AD, to the extent that individual has AD, whether or not one or more non-AD neurodegenerative diseases or conditions are previously, concurrently or subsequently diagnosed.

The compounds and methods of the present invention are useful for individuals who have received prior medication for AD, as well as individuals who have received no prior medication for AD.

Individuals of any age may be treated by the methods of the invention, with the pharmaceutical compositions of the invention; however, the invention encompasses specific embodiments for treating or preventing Alzheimer's disease in individuals between the ages of 45 and 100. In other various specific embodiments, individuals treated by the therapeutic or prophylactic methods of the invention may be from 55 to 70 years of age, 60 to 80 years of age, 55 to 65 years of age, 60 to 75 years of age, 65 to 80 years of age, 55 to 60 years of age, 60 to 65 years of age, 65 to 70 years of age, 70 to 75 years of age, 75 to 80 years of age, or 80 years old and older.

Additionally, the invention, is some embodiments, relates to identifying an individual who is experiencing a decrease in the ratio of Aβ₄₂/Aβ₄₀ ratio in cerebral spinal fluids (CSF) levels and treating said individual with a combination of the amount of one or more Aβ₄₂ lowering agents and one or more hormonal modulating agents. Method of monitoring CSF levels of Aβ₄₂ and Aβ₄₀ are known to the skilled artisan and described herein.

Thus, in one embodiment, the invention provides a method of treating an individual known or suspected of having Alzheimer's disease comprising administering an effective amount of an Aβ₄₂ lowering agent and a steroidal anti-inflammatory agent. In a specific embodiment, said individual is diagnosed as having mild to moderate Alzheimer's disease. In another specific embodiment, said individual is diagnosed by a cognitive test as having mild to moderate AD. In yet another specific embodiment, the cognitive test is the Mini-Mental State Exam (MMSE). In an even more specific embodiment, the individual has a score in the MMSE of from 26 to 19, inclusive. In another more specific embodiment, the individual has a score in the MMSE of from 18 to 10, inclusive. In another specific embodiment, the individual has a score in said MMSE of 26 to 10, inclusive.

Cancer patients and individuals desiring cancer prophylaxis can be treated with the combinations of the invention.

The terms “cancer” and “tumor” refer to or describe the physiological condition in mammals that is typically characterized by unregulated cell growth. By means of the pharmaceutical co-formulations and combination treatments of the present invention, various tumors can be treated such as tumors of the breast, heart, lung, small intestine, colon, spleen, kidney, bladder, head and neck, ovary, prostate, brain, pancreas, skin, bone, bone marrow, blood, thymus, uterus, testicles, cervix, and liver. In one embodiment, the tumor or cancer is chosen from adenoma, angio-sarcoma, astrocytoma, epithelial carcinoma, germinoma, glioblastoma, glioma, hamartoma, hemangioendothelioma, hemangiosarcoma, hematoma, hepato-blastoma, leukemia, lymphoma, medulloblastoma, melanoma, neuroblastoma, osteosarcoma, retinoblastoma, rhabdomyosarcoma, sarcoma, and teratoma. The tumor can be chosen from acral lentiginous melanoma, actinic keratoses, adenocarcinoma, adenoid cycstic carcinoma, adenomas, adenosarcoma, adenosquamous carcinoma, astrocytic tumors, bartholin gland carcinoma, basal cell carcinoma, bronchial gland carcinomas, capillary, carcinoids, carcinoma, carcinosarcoma, cavernous, cholangio-carcinoma, chondosarcoma, choriod plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic, papillary serous adeno-carcinoma, pineal cell, pituitary tumors, plasmacytoma, pseudo-sarcoma, pulmonary blastoma, renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiated carcinoma, uveal melanoma, verrucous carcinoma, vipoma, well differentiated carcinoma, and Wilm's tumor.

In one embodiment, the combination treatment and/or co-formulation is used to treat prostate cancer. In one aspect of this embodiment, the primary prostate cancer was treated with surgery and/or radiotherapy, and the patient is at risk for having a prostate cancer recurrence.

In some embodiments, the combination treatments and co-formulation are useful for treating acute myelogenous leukemia (AML), chronic myelogenous leukemia (CML), acute lymphocytic leukemia (ALL), chronic lymphocytic leukemia (CLL), hairy cell leukemia (HCL) and all subtypes of these leukemias which are defined by morphological, histochemical and immunological techniques that are well known by those skilled in the art.

In another embodiment, the present invention provides a method for treating myelogenous leukemia by administering to the patient a therapeutically effective amount of one or more Aβ₄₂ lowering agents and one or more hormonal modulating agents.

In another embodiment, the present invention provides a method for treating acute myelogenous leukemia by administering to the patient a therapeutically effective amount of one or more Aβ₄₂ lowering agents and one or more hormonal modulating agents.

In another embodiment, the present invention provides a method for treating chronic myelogenous leukemia by administering to the patient a therapeutically effective amount of one or more Aβ₄₂ lowering agents and one or more hormonal modulating agents.

In another embodiment, the present invention provides a method for treating chronic myelogenous leukemia in blastic phase by administering to the patient a therapeutically effective amount of one or more Aβ₄₂ lowering agents and one or more hormonal modulating agents.

In another embodiment, the present invention provides a method for treating refractory/relapsed leukemia by administering to the patient a therapeutically effective amount of one or more Aβ₄₂ lowering agents and one or more hormonal modulating agents.

Specific leukemias that can be treated with the combinations of the invention include, but are not limited to, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

Lymphomas can be treated with the combinations of the invention. Lymphomas are generally neoplastic transformations of cells that reside primarily in lymphoid tissue. Among lymphomas, there are two major distinct groups: non-Hodgkin's lymphoma (NHL) and Hodgkin's disease. Lymphomas are tumors of the immune system and generally are present as both T cell- and as B cell-associated disease. Bone marrow, lymph nodes, spleen and circulating cells are all typically involved. Treatment protocols include removal of bone marrow from the patient and purging it of tumor cells, often using antibodies directed against antigens present on the tumor cell type, followed by storage. The patient is then given a toxic dose of radiation or chemotherapy and the purged bone marrow is then re-infused in order to repopulate the patient's hematopoietic system.

Other hematological malignancies that can be treated with the combinations of the invention include myelodysplastic syndromes (MDS), myeloproliferative syndromes (MPS) and myelomas, such as solitary myeloma and multiple myeloma. Multiple myeloma (also called plasma cell myeloma) involves the skeletal system and is characterized by multiple tumorous masses of neoplastic plasma cells scattered throughout that system. It may also spread to lymph nodes and other sites such as the skin. Solitary myeloma involves solitary lesions that tend to occur in the same locations as multiple myeloma.

Additional Combination Therapy

The invention further provides additional combination therapy strategies for treating neurodegenerative disorders such as Alzheimer's disease, MCI, and dementia. According to this aspect of the invention, an individual in need of treatment is administered an effective amount of (1)one or more Aβ₄₂ lowering agents, (2) one or more steroidal agents, and (3) one or more compounds selected from the group consisting of NSAIDs, acetylcholine esterase inhibitors (e.g., donepezil, galantamine, rivastagmine), COX-2 inhibitors (cyclooxygenase-2), β-secretase inhibitors, γ-secretase inhibitors, NMDA antagonists (i.e., memantine), and GABA-A alpha inverse agonist (see WO 00/27382, WO 96/25948, WO 98/50385 which are herein incorporated by reference in there entireties). NMDA receptor antagonists for combination therapy are memantine, adamantane, amantadine, an adamantane derivative, dextromethorphan, dextrorphan, dizocilpine, ibogaine, ketamine, and remacemide. The combination therapy of the invention is thought to provide a synergistic effect in reducing Aβ₄₂ levels and is surprisingly thought to be especially effective for treating and preventing neurodegenerative disorders including Alzheimer's disease, dementia, and MCI. The invention further encompasses compositions comprising the combination of active ingredients of this aspect of the invention.

Pharmaceutical Dosage Forms and Their Preparation

The unit dosage forms of the co-formulations of the invention can be formulated for oral administration, although in some embodiments combination treatments may involve other routes of administration such as parenteral routes like intravenous, subcutaneous, intramuscular, intraperitoneal, and intramammary routes.

Oral pharmaceutical dosage forms can be in the form of a solid, gel or liquid. Examples of solid dosage forms include, but are not limited to, tablets, capsules, granules, and bulk powders. Specific examples of oral tablets include compressed tablets, chewable lozenges, and tablets that can be enteric-coated, sugar-coated or film-coated. Specific examples of capsules include hard and soft gelatin capsules. Granules and powders can be provided in non-effervescent and effervescent forms. Each can be combined with other ingredients known to those skilled in the art.

Capsules are dosage forms where the API is enclosed in a shell or container usually made of gelatin. Soft gelatin capsules can be prepared in which capsules contain a mixture of the API and vegetable oil or non-aqueous, water miscible materials such as, for example, polyethylene glycol and the like. Hard gelatin capsules may contain granules of the API in combination with a solid, pulverulent carrier, such as, for example, lactose, saccharose, sorbitol, mannitol, potato starch, corn starch, amylopectin, cellulose derivatives, or gelatin. Additionally, API can be present in the gelatin capsule shell.

Examples of liquid oral dosage forms that can be used include, but are not limited to, aqueous solutions, emulsions, suspensions, solutions and/or suspensions reconstituted from non-effervescent granules and effervescent preparations reconstituted from effervescent granules.

Examples of aqueous solutions that can be used include, but are not limited to, elixirs and syrups. As used herein, elixirs refer to clear, sweetened, hydroalcoholic preparations. Examples of pharmaceutically acceptable carriers that may be used in elixirs include, but are not limited to solvents. Particular examples of solvents that may be used include glycerin, sorbitol, ethyl alcohol, and syrup. As used herein, syrups refer to concentrated aqueous solutions of a sugar, for example, sucrose. Syrups may optionally further comprise a preservative.

Emulsions refer to two-phase systems in which one liquid is dispersed in the form of small globules throughout another liquid. In some embodiments, emulsions can be oil-in-water or water-in-oil emulsions. Examples of pharmaceutically acceptable carriers that may be used in emulsions include, but are not limited to non-aqueous liquids, emulsifying agents, and preservatives.

Examples of pharmaceutically acceptable substances that can be used in non-effervescent granules, to be reconstituted into a liquid oral dosage form, include diluents, sweeteners and wetting agents.

Examples of pharmaceutically acceptable substances that can be used in effervescent granules, to be reconstituted into a liquid oral dosage form, include organic acids and a source of carbon dioxide.

Tablet Preparation

In general, there are three general methods of tablet preparation: (1) the wet-granulation method; (2) the dry-granulation method; and (3) direct compression. These methods are well known to those skilled in the art. See, Remington's Pharmaceutical Sciences, 16th and 18th Eds., Mack Publishing Co., Easton, Pa. (1980 and 1990). See, also, U.S. Pharmacopeia XXI, U.S. Pharmacopeial Convention, Inc., Rockville, Md. (1985).

In one embodiment, the co-formulation tablets of the invention can be manufactured using a high shear wet granulation method incorporating pre-blending and pre-milling to reduce the size of the large particles in the drug substance. Once granulated, the material can be dried, milled, and blended again. The final powder blend can be compressed into tablets on a high-speed rotary press and the resulting tablets coated in a perforated pan.

Tablets for oral use in the methods of the invention can be prepared in the following manner, although other techniques can be employed. The solid substances are ground or sieved to a desired particle size, and the binding agent is homogenized and suspended in a suitable solvent. The active pharmaceutical ingredient(s) and auxiliary agents are mixed with the binding agent solution. The resulting mixture is moistened to form a uniform suspension. The moistening typically causes the particles to aggregate slightly, and the resulting mass is gently pressed through a stainless steel sieve having a desired size. The layers of the mixture are then dried in controlled drying units for determined length of time to achieve a desired particle size and consistency. The granules of the dried mixture are gently sieved to remove any powder. To this mixture, disintegrating, anti-friction, and anti-adhesive agents are added. Finally, the mixture is pressed into tablets using a machine with the appropriate punches and dies to obtain the desired tablet size. The operating parameters of the machine may be selected by the skilled artisan.

Various tablet formulations can be made in accordance with the present invention. These include tablet dosage forms such as sugar-coated tablets, film-coated tablets, enteric-coated tablets, multiple-compressed tablets, prolonged action tablets and the like. Sugar-coated tablets (SCT) are compressed tablets containing a sugar coating. Such coatings may be colored and are beneficial in covering up drug substances possessing objectionable tastes or odors and in protecting materials sensitive to oxidation. Film-coated tablets (FCT) are compressed tablets that are covered with a thin layer or film of a water-soluble material. A number of polymeric substances with film-forming properties may be used. The film coating imparts the same general characteristics as sugar coating with the added advantage of a greatly reduced time period required for the coating operation. Enteric-coated tablets are also suitable for use in the present invention. Enteric-coated tablets (ECT) are compressed tablets coated with substances that resist dissolution in gastric fluid but disintegrate in the intestine. Enteric coating can be used for tablets containing drug substances that are inactivated or destroyed in the stomach, for those which irritate the mucosa or as a means of delayed release of the medication.

Multiple compressed tablets (MCT) are compressed tablets made by more than one compression cycle, such as layered tablets or press-coated tablets. Layered tablets are prepared by compressing additional tablet granulation on a previously compressed granulation. The operation may be repeated to produce multilayered tablets of two, three or more layers. Typically, special tablet presses are required to make layered tablets. See, e.g., U.S. Pat. No. 5,213,738, incorporated herein in its entirety by reference thereto.

Press coated tablets are another form of multiple compressed tablets. Such tablets, also referred to as dry-coated tablets, are prepared by feeding previously compressed tablets into a tableting machine and compressing another granulation layer around the preformed tablets. These tablets have all the advantages of compressed tablets, i.e., slotting, monogramming, speed of disintegration, etc., while retaining the attributes of sugar coated tablets in masking the taste of the drug substance in the core tablet. Press-coated tablets can also be used to separate incompatible drug substances. Further, they can be used to provide an enteric coating to the core tablets. Both types of tablets (i.e., layered tablets and press-coated tablets) may be used, for example, in the design of prolonged-action dosage forms of the present invention.

Pharmaceutical compositions or unit dosage forms of the present invention in the form of prolonged-action tablets may comprise compressed tablets formulated to release the drug substance in a manner to provide medication over a period of time. There are a number of tablet types that include delayed-action tablets in which the release of the drug substance is prevented for an interval of time after administration or until certain physiological conditions exist. Repeat action tablets may be formed that periodically release a complete dose of the drug substance to the gastrointestinal fluids. Also, extended release tablets that continuously release increments of the contained drug substance to the gastrointestinal fluids may be formed.

In practical use, the combination of one or more Aβ₄₂ lowering agents and one or more hormonal modulating agents can be combined as the active pharmaceutical ingredient in intimate admixture with a pharmaceutically acceptable carrier according to conventional pharmaceutical compounding techniques. The pharmaceutically acceptable carrier may take a wide variety of forms depending on the form of preparation desired for administration, e.g., oral, parenteral (including intravenous, subcutaneous, intrathecal, and intramuscular), transdermal, and topical. In preparing the compositions for oral dosage form, any of the usual pharmaceutical media or excipients may be employed. These include, for example, water, glycols, oils, alcohols, flavoring agents, preservatives, coloring agents and the like in the case of oral liquid preparations such as suspensions, elixirs and solutions; or aerosols; or excipients such as starches, sugars, microcrystalline cellulose, diluents, granulating agents, lubricants, binders, disintegrating agents and the like in the case of oral solid preparations such as powders, capsules, caplets, and tablets. Solid oral preparations are generally preferred over liquid ones. Because of their ease of administration, tablets and capsules represent the most advantageous oral dosage unit forms, in which case solid pharmaceutical pharmaceutically acceptable excipients are obviously employed. If desired, tablets may be coated by standard aqueous or nonaqueous techniques. Preferred solid oral preparations are tablets and capsules.

Pharmaceutical stabilizers may be used to stabilize compositions of the invention, or pharmaceutically acceptable salts, solvates, or clathrates thereof. Acceptable stabilizers include, but are not limited to, L-cysteine hydrochloride, glycine hydrochloride, malic acid, sodium metabisulfite, citric acid, tartaric acid, and L-cystine dihydrochloride. See, e.g., U.S. Pat. Nos.: 5,731,000; 5,763,493; 5,541,231; and 5,358,970, all of which are incorporated herein by reference.

Inactive Pharmaceutical Ingredients

The formulations and unit dosage forms of the invention can have a number of different ingredients. Depending on the dosage strength, a unit dosage form has an amount of active pharmaceutical ingredient(s) (API) sufficient for achieving a therapeutic effect in a target population. Additionally “inactive pharmaceutical ingredients” need to be present to achieve a therapeutically effective release of the API. Thus the amount and type of inactive ingredients help achieve a therapeutically effective release of the therapeutic agent. In one aspect of the invention, a tablet unit dosage form is provided having the following inactive ingredients: one or more disintegrants in an amount sufficient to facilitate break-up (disintegration) of the tablet after administration (e.g., provide an immediate release dissolution profile), one or more binders in an amount sufficient to impart adequate cohesiveness to the tablet and/or provide adequate free flowing qualities by formulation of granules of desired size/hardness, one or more diluents in an amount sufficient to impart satisfactory compression characteristics, one or more lubricants in an amount sufficient to provide an adequate flow rate of the granulation and/or prevent adhesion of the material to the die/punch, reduce interparticle friction, and/or facilitate ejection from the die, and if desired, optional ingredients.

The disintegration rate, and often the dissolution rate of a compacted solid pharmaceutical formulation in an aqueous environment (e.g., the patient's stomach) may be increased by the addition of a disintegrant to the formulation. Disintegrants include alginic acid, carboxymethylcellulose calcium, carboxymethylcellulose sodium (e.g., Ac-Di-Sol® Primellose®.), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®) and starch.

Solid pharmaceutical formulations that are compacted into a dosage form, such as a tablet, can include excipients whose functions include helping to bind the active pharmaceutical ingredient and other excipients together after compression. Binders for solid pharmaceutical formulations include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methylcellulose (e.g. Methocel®), lactose, liquid glucose, magnesium aluminum silicate, maltodextrin, methylcellulose, polymethacrylates, povidone (e.g. Kollidon®, Plasdone®), pregelatinized starch, sodium alginate and starch. Glidants can be added to improve the flowability of a non-compacted solid formulation and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered formulation, the formulation is subjected to pressure from a punch and dye. Some excipients and active pharmaceutical ingredients have a tendency to adhere to the surfaces of the punch and dye, which can cause the product to have pitting and other surface irregularities. A lubricant can be added to the formulation to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Examples of diluents include, but are not limited to, calcium carbonate, calcium phosphate, calcium sulfate, cellulose, cellulose acetate, compressible sugar, confectioner's sugar, dextrates, dextrin, dextrose, ethyl cellulose, fructose, fumaric acid, glyceryl palmitostearate, hydrogenated vegetable oil, kaolin, lactitol, lactose, magnesium carbonate, magnesium oxide, maltodextrin, maltose, mannitol, medium chaim glyceride, microcrystalline cellulose, polydextrose, polymethylacrylates, simethicone, sodium alginate, sodium chloride, sorbitol, starch, pregelantized starch, sterilizable maize, sucrose, sugar spheres, talc, tragacanth, trehalose, and xylitol.

Examples of disintegrants include, but are not limited to, alginic acid, calcium phosphate, carboxymethyl cellulose calcium, carboxymethyl cellulose sodium, powdered cellulose, chitosan, crospovidone, docusate sodium, guar gum, hydroxylpropyl cellulose, magnesium aluminum silicate, methylcellulose, poidone, sodium alginate, sodium starch glycolate, starch, and pregelantinized starch.

Example of binders (binding agents) include, but are not limited to, acacia, alginic acid, carbomers, carboxymethyl cellulose sodium, carrageenan, cellulose acetate phthalate, ceratonia, chitosan, confectioners sugar, cottonseed oil, dextrates, dextrin, dextrose, ethylcellulose, gelatin, glucose, glyceryl behenate, guar gum, hydrogenated vegetable oil, hydroxyethyl cellulose, hydroxyethylmethyl cellulose, hydroxylpropyl cellulose, hypromellose, magnesium aluminum silicate, maltodextrin, maltodextrin, maltose, methylcellulose, microcrystalline cellulose, poloxamer, polydextrose, polyethylene oxide, polymethyl acrylates, povidone, sodium alginate, starch, pregelantized starch, stearic acid, sucrose, sunflower oil, and zein.

Examples of lubricants include, but are not limited to, calcium stearate, glycerin monostearate, glyceryl behenate, glyceryl palmitostearate, hydrogenated castor oil, hydrogenated vegetable oil, light mineral oil, magnesium lauryl sulfate, magnesium stearate, medium chain triglycerides, mineral oil, poloxamer, polyethylene glycol, sodium benzoate, sodium chloride, sodium lauryl sulfate, sodium stearyl fumarate, stearic acid, talc, and zinc stearate.

Examples of glidants include, but are not limited to, calcium phosphate, calcium silicate, cellulose powdered, colloidal silicon dioxide, magnesium silicate, magnesium trisilicate, silicon dioxide, starch, and talc.

Optional ingredients in the formulations of the invention include, but are not limited to, flavors, coloring agents, and stabilizers.

Flavoring agents and flavor enhancers make the dosage form more palatable to the patient. Common flavoring agents and flavor enhancers for pharmaceutical products that may be included in the formulation of the present invention include maltol, vanillin, ethyl vanillin, menthol, citric acid, fumaric acid, ethyl maltol and tartaric acid.

Solid and liquid formulations may also be dyed using any pharmaceutically acceptable colorant to improve their appearance and/or facilitate patient identification of the product and unit dosage level.

In one embodiment, the tablet unit dosage form has a hardness of about 5 kp (kilopond) or more, about 7 kp or more, about 9 kp or more, about 11 kp or more, and about 13 kp or more to avoid excessive friability, and a hardness of about 20 kp or less, about 19 kp or less, about 18 kp or less, about 17 kp or less, and about 16 kp or less, is desirable to avoid subsequent difficulty in hydrating the tablet when exposed to gastric fluid. In some aspects of this embodiment, the hardness of the tablet unit dosage form is from 9 kp to 18 kp, 11 kp to 17 kp, and 13 kp to 17 kp. When hardness is in an acceptable range, tablet friability is typically less than about 1.0%, preferably less than about 0.8% and more preferably less than about 0.5%, in a standard test. While the skilled artisan recognizes that there are numerous techniques available for determining hardness, for purposes of comparison, the method used to determine tablet hardness of the unit dosage forms of the invention (as described in Example 6) should be used. Some issues that may cause variations in tablet hardness are inconsistent tablet weight, particle size variations, poor powder compressibility, and insufficient binder level.

One problem encountered with tablet unit dosage forms is that they can often exhibit high friability. Friability is a physical parameter of a solid dosage form that relates to the tablets ability to withstand physical perturbations. Friability is the tendency of a tablet to crumble, chip or break. Dosage forms having a high friability will rapidly dissolve or disintegrate. An optimum unit dosage form will rapidly dissolve or disintegrate and have a low level of friability. The tablets of the invention have a friability of less than about 1%, meaning that the tablets meet the United States Pharmacopeia standard for tablet friability (which requires a friability of less than 1%). Friable tablets can be caused by low moisture content, insufficient binder, tablet configuration (e.g., sharp versus beveled edges).

The tablet unit dosage forms of the invention have a friability of less than about 1%, less than about 0.9%, less than about 0.8%, less than about 0.7%, less than about 0.6%, less than about 0.5%, and less than about 0.4% (all at 100 rev).

Poor disintegration can come from tablets which are compressed too hard, insufficient disintegrant levels, or too much binder.

Pharmaceutically Acceptable Salts

“Pharmaceutically-acceptable salt” refers to salts of the compounds of for use in the invention, which are substantially non-toxic to mammals. Typical pharmaceutically-acceptable salts include those salts prepared by reaction of the compounds of the present invention with a mineral or organic acid or an organic or inorganic base. Such salts are known as acid or base addition salts, respectively. It should be recognized that the particular counterion forming a part of any salt of this invention is not of a critical nature, so long as the salt as a whole is pharmaceutically-acceptable and as long as the counterion does not contribute undesired qualities to the salt as a whole.

In some embodiments, the compounds of the invention are acids (e.g., (R)-2-(2-fluoro-4-biphenylyl)propionic acid) and form salts with pharmaceutically acceptable bases. Some examples of base addition salts include metal salts such as aluminum; alkali metal salts such as lithium, sodium or potassium; and alkaline earth metal salts such as calcium, magnesium, ammonium, or substituted ammonium salts. Examples of substituted ammonium salts include, for instance, those with lower alkylamines such as trimethylamine, triethylamine; hydroxyalkylamines such as 2-hydroxyethylamine, bis-(2-hydroxyethyl)-amine or tri-(2-hydroxyethyl)-amine, cycloalkylamines such as bicyclohexylamine or dibenzylpiperidine, N-benzyl-p-β-phenethylamine, dehydroabietylamine, N,N′-bisdehydro-abietylamine, glucamine, N-methylglucamine; bases of the pyridine type such as pyridine, collidine, quinine or quinoline; and salts of basic amino acids such as lysine and arginine.

Examples of inorganic bases include, without limitation, sodium hydroxide, potassium hydroxide, potassium carbonate, sodium carbonate, sodium bicarbonate, potassium bicarbonate, calcium hydroxide, calcium carbonate, and the like.

In one embodiment, the pharmaceutically acceptable salt is chosen from ammonium, D-arginine, L-arginine, D,L-arginine, t-butylamine, calcium, choline, diethanolamine, ethylenediamine, glycine, L-histidine, D-lysine, L-lysine, D,L-lysine, magnesium, N-methyl-D-glucamine, L-ornithine hydrochloride, potassium, procaine hydrochloride, L-proline, pyridoxine, L-serine, sodium, DL-tryptophan, L-tryptophan, tromethamine, L-tyrosine, L-valine, and lithium.

### EXAMPLES

### Example 1

Co-formulation of (R)-2-(2-fluoro-4-biphenylyl)propionic Acid with a Steroidal Anti-Inflammatory Agent.

### 2-(2-Fluoro-4-biphenylyl)propionic Acid Dexamethasone

| Ingredient | Amount |
|---|---|
| (R)-2-(2-Fluoro-4-biphenylyl)propionic | 400 mg |
| Acid | |
| Microcrystalline Cellulose | 392 mg |
| Colloidal Silicon Dioxide | 4 mg |
| Magnesium Stearate | 4 mg |
| Dexamethasone | 1 mg |

### (R)-2-(2-Fluoro-4-biphenylyl)propionic Acid Prednisolone Tablets

| Ingredient | Amount |
|---|---|
| (R)-2-(2-Fluoro-4-biphenylyl)propionic | 400 mg |
| Acid | |
| Microcrystalline Cellulose | 392 mg |
| Colloidal Silicon Dioxide | 4 mg |
| Magnesium Stearate | 4 mg |
| Prednisolone | 25 mg |

### (R)-2-(2-Fluoro-4-biphenylyl)propionic Acid Prednisone Tablets

| Ingredient | Amount |
|---|---|
| (R)-2-(2-Fluoro-4-biphenylyl)propionic | 400 mg |
| Acid | |
| Microcrystalline Cellulose | 392 mg |
| Colloidal Silicon Dioxide | 4 mg |
| Magnesium Stearate | 4 mg |
| Prednisone | 25 mg |

### (R)-2-(2-Fluoro-4-biphenylyl)propionic Acid Cortisone Tablets

| Ingredient | Amount |
|---|---|
| (R)-2-(2-Fluoro-4-biphenylyl)propionic | 400 mg |
| Acid | |
| Microcrystalline Cellulose | 392 mg |
| Colloidal Silicon Dioxide | 4 mg |
| Magnesium Stearate | 4 mg |
| Cortisone | 25 mg |

The tablets are prepared using art known procedures and the amounts ingredients listed above can be modified (e.g., coated) to obtain an improved formulation.

### Example 2

Co-formulation of 2-(4-isobutyl-phenyl)-2-methyl Propionic Acid and a Steroidal Anti-inflammatory Agent.

### 2-(4-Isobutyl-phenyl)-2-methyl Propionic Acid Dexamethasone Tablets

| Ingredient | Amount |
|---|---|
| 2-(4-isobutyl-phenyl)-2-methyl | 400 mg |
| propionic acid | |
| Microcrystalline Cellulose | 392 mg |
| Colloidal Silicon Dioxide | 4 mg |
| Magnesium Stearate | 4 mg |
| Dexamethasone | 1 mg |

### 2-(4-Isobutyl-phenyl)-2-methyl Propionic Acid Prednisolone Tablets

| Ingredient | Amount |
|---|---|
| (R)-2-(2-Fluoro-4-biphenylyl)propionic | 400 mg |
| Acid | |
| Microcrystalline Cellulose | 392 mg |
| Colloidal Silicon Dioxide | 4 mg |
| Magnesium Stearate | 4 mg |
| Prednisolone | 25 mg |

### 2-(4-Isobutyl-phenyl)-2-methyl Propionic Acid Prednisone

| Ingredient | Amount |
|---|---|
| 2-(4-Isobutyl-phenyl)-2-methyl | 400 mg |
| Propionic Acid | |
| Microcrystalline Cellulose | 392 mg |
| Colloidal Silicon Dioxide | 4 mg |
| Magnesium Stearate | 4 mg |
| Prednisone | 25 mg |

2-(4-Isobutyl-phenyl)-2-methyl Propionic Acid Cortisone Tablets

| Ingredient | Amount |
|---|---|
| 2-(4-Isobutyl-phenyl)-2-methyl | 400 mg |
| Propionic Acid | |
| Microcrystalline Cellulose | 392 mg |
| Colloidal Silicon Dioxide | 4 mg |
| Magnesium Stearate | 4 mg |
| Cortisone | 25 mg |

### Example 3

Co-formulation of 2-(2-fluoro- 1,1′-biphenyl-4-yl)-2-methylpropionic Acid With a Steroidal Anti-inflammatory Agent

### 2-(2-Fluoro-1,1′-biphenyl-4-yl)-2-methylpropionic Acid Dexamethasone Tablets

| Ingredient | Amount |
|---|---|
| 2-(2-Fluoro-1,1′-biphenyl-4-yl)-2- | 400 mg |
| methylpropionic Acid | |
| Microcrystalline Cellulose | 392 mg |
| Colloidal Silicon Dioxide | 4 mg |
| Magnesium Stearate | 4 mg |
| Dexamethasone | 1 mg |

### 2-(2-Fluoro-1,1′-biphenyl-4-yl)-2-methylpropionic Acid Prednisolone Tablets

| Ingredient | Amount |
|---|---|
| 2-(2-Fluoro-1,1′-biphenyl-4-yl)-2- | 400 mg |
| methylpropionic Acid | |
| Microcrystalline Cellulose | 392 mg |
| Colloidal Silicon Dioxide | 4 mg |
| Magnesium Stearate | 4 mg |
| Prednisolone | 25 mg |

### 2-(2-Fluoro-1,1′-biphenyl-4-yl)-2-methylpropionic Acid Prednisone Tablets

| Ingredient | Amount | |
|---|---|---|
| 2-(2-Fluoro-1,1′-biphenyl-4-yl)-2- | 400 | mg |
| methylpropionic Acid | | |
| Microcrystalline Cellulose | 392 | mg |
| Colloidal Silicon Dioxide | 4 | mg |
| Magnesium Stearate | 4 | mg |
| Prednisone | 25 | mg |

### 2-(2-Fluoro-1,1′-biphenyl-4-yl)-2-methylpropionic Acid Cortisone Tablets

| Ingredient | Amount | |
|---|---|---|
| 2-(2-Fluoro-1,1′-biphenyl-4-yl)-2- | 400 | mg |
| methylpropionic Acid | | |
| Microcrystalline Cellulose | 392 | mg |
| Colloidal Silicon Dioxide | 4 | mg |
| Magnesium Stearate | 4 | mg |
| Cortisone | 25 | mg |

### Example 2

Treatment of Alzheimer's Disease with (R)-2-(2-Fluoro-4-biphenylyl)propionic Acid and a Steroidal Anti-inflammatory Agent

The (R)-2-(2-fluoro-4-biphenylyl)propionic acid can be administered twice daily as tablets containing a total of 800 mg (1600 mg/day) of active ingredient (one, two, or three or more tablets) or as a capsule containing 800 mg of the active ingredient (one, two, or three or more capsules). A higher dose can be administered to the patient in need of such treatment which can involve the patient taking, e.g., a 1000 mg dose of (R)-2-(2-fluoro-4-biphenylyl)propionic acid in the morning and a 1000 mg dose of (R)-2-(2-fluoro-4-biphenylyl)propionic acid in the evening. Dexamethasone can be administered twice daily as tablets containing 1.5 mg of dexamethasone (or 1 mg twice daily). The (R)-2-(2-fluoro-4-biphenylyl)propionic acid and dexamethasone can also be co-formulated.

Typically, for the treatment of mild-to-moderate Alzheimer's disease, an individual is diagnosed by a doctor as having the disease using a suitable combination of observations. One criterion indicating a likelihood of mild-to-moderate Alzheimer's disease is a score of about 15 to about 26 on the MMSE test (in a specific sub-group the patient has an MMSE of from 20-26, inclusive). Another criteria indicating mild-to-moderate Alzheimer's disease is a decline in cognitive function.

Depending on the stage of the disease, (R)-2-(2-fluoro-4-biphenylyl)propionic acid can also be administered twice daily in liquid, capsule, or tablet dosage forms where the dose has various amounts of (R)-2-(2-fluoro-4-biphenylyl)propionic acid (i.e., 850 mg, 750 mg, 700 mg, 650 mg, 600 mg, 550 mg, 500 mg, 450 mg, 350 mg, 300 mg, 250 mg, 200 mg, 150 mg, and 100 mg). Again, the dosages can also be divided or modified, and taken with or without food.

Patients having mild-to-moderate Alzheimer's disease undergoing the treatment regimen of this example with (R)-2-(2-fluoro-4-biphenylyl)propionic acid doses of about 800 mg (BID) and dexamethasone 1.5 mg BID (or 1 mg BID) can experience a lessening in decline of cognitive function (as measured by the ADAS-cog or CDR sum of boxes), plaque pathology, and/or biochemical disease marker progression.

### Example 3

Detection of Amyloid Beta with Biosource Elisa Kit (Camarillo, Calif.)

The present invention provides combination compositions and methods for lowering Aβ₄₂ levels. To test whether the combinations are capable of modulating Aβ levels, a sandwich enzyme-linked immunosorbent assay (ELISA) is employed to measure secreted Aβ (Aβ₄₂ and/or Aβ₄₀) levels. In this example, H4 cells expressing wide type APP695 are seeded at 200,000 cells/ per well in 6 well plates, and incubated at 37 ° C. with 5% CO₂ overnight. Cells are treated with 1.5 ml medium containing vehicle (DMSO) or a test compounds at 1.25 μM, 2.5 μM, 5.0 μM and 10.0 μM (as well as other concentration if desirable) concentration for 24 hours or 48 hours. The supernatant from treated cells is collected into eppendorf tubes and frozen at −80 ° C. for future analysis.

The amyloid peptide standard is reconstituted and frozen samples are thawed. The samples and standards are diluted with appropriate diluents and the plate is washed 4 times with Working Wash Buffer and patted dry on a paper towel. 100 μL per well of peptide standards, controls, and dilutions of samples to be analyzed is added. The plate is incubated for 2 hours while shaking on an orbital plate shaker at RT. The plate is then washed 4 times with Working Wash Buffer and patted dry on a paper towel. Detection Antibody Solution is poured into a reservoir and 100 μL /well of Detection Antibody Solution is immediately added to the plate. The plate is incubated at RT for 2 hours while shaking and then washed four times with Working Wash Buffer and patted dry on a paper towel. Secondary Antibody Solution is then poured into a reservoir and 100 μL /well of Secondary Antibody Solution is immediately added to the plate. The plate is incubated at RT for 2 hours with shaking, washed 5 times with Working Wash Buffer, and patted dry on a paper towel.

100 μL of stabilized chromogen is added to each well and the liquid in the wells begins to turn blue. The plate is incubated for 30 minutes at room temperature and in the dark. 100 μL of stop solution is added to each well and the plate is tapped gently to mix resulting in a change of solution color from blue to yellow. The absorbance of each well is read at 450 nm having blanked the plate reader against a chromogen blank composed of 100 μL each of stabilized chromogen and stop solution. The plate is read within 2 hours of adding the stop solution. The absorbance of the standards is plotted against the standard concentration and the concentrations of unknown samples and controls are calculated.

### Example 4

Combination Treatment of Animals to Determine the Combination's Effect on Memory and Alzheimer's Disease Progression.

The present invention provides combination compositions and methods for treating or preventing Alzheimer's disease. To test the effect of compositions of the present invention on memory and Alzheimer's disease, TG2576 mice that overexpress APP(695) with the “Swedish” mutation (APP695NL) are used. Mice overexpressing APP(695) with the “Swedish” mutation develop memory deficits and plaques with age, making them suitable for examining the effect of compounds ((R)-2-(2-fluoro-4-biphenylyl)propionic acid and dexamethasone) on memory and Alzheimer's Disease. The test compounds are administered daily for two weeks to test groups of the TG2576 mice in age groups of: 1) 4-5 months, 2) 6-11 months, 3) 12-18 months, and 4) 20-25 months. Groups of control TG2576 mice of corresponding ages are not administered the compound. Both control and test groups then have memory tested in a version of the Morris water maze (Morris, J. Neurosci. Methods, 11:47-60 (1984)) that is modified for mice. The water maze contains a metal circular pool of about 40 cm in height and 75 cm in diameter. The walls of the pool have fixed spatial orientation clues of distinct patterns or shelves containing objects. The pool is filled with room temperature water to a depth of 25cm and an escape platform is hidden 0.5 cm below the surface of the 25-cm-deep water at a fixed position in the center of one of the southwest quadrant of pool. The test and control mice are trained for 10 days in daily sessions consisting of four trials in which the mouse starts in a different quadrant of the pool for each trial. The mice are timed and given 60 seconds to find the escape platform in the pool. If the mice have not found the escape platform after 60 seconds, they are guided into it. The mice are then allowed to rest on the platform for 30 seconds and the amount of time it takes the mice to find the platform is recorded. Probe trials are run at the end of the trials on the 4th, 7th, and 10th days of training, in which the platform is removed and the mice are allowed to search for the platform for 60 sec. The percentage of time spent in the quadrant where the platform was in previous trials is calculated.

In training trials, the time it takes test group mice to reach the escape platform is compared to the time taken by control group mice of corresponding ages. In probe trials, the percentage of time spent by test group mice in the quadrant where the platform was in previous trials is compared to the percentage time spent by control mice. Quicker location of the escape platform in training trials and/or an increased percentage time spent in the previous quadrant of the maze during probe trials is indicative of spatial learning and memory. Because memory loss is a hallmark of Alzheimer's disease, test mice that have better learning and memory when compared to control mice indicate that the combination can be effective in treating or slowing Alzheimer's disease and/or its symptoms.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

What is claimed is:

1. A co-formulation having a therapeutically effective amount one or more Aβ₄₂ lowering agents and one or more hormonal modulating agents, and one or more pharmaceutically acceptable excipients.

2. The co-formulation of claim 1 wherein said one or more Aβ₄₂ lowering agents is chosen from a compound having one of the following formulae 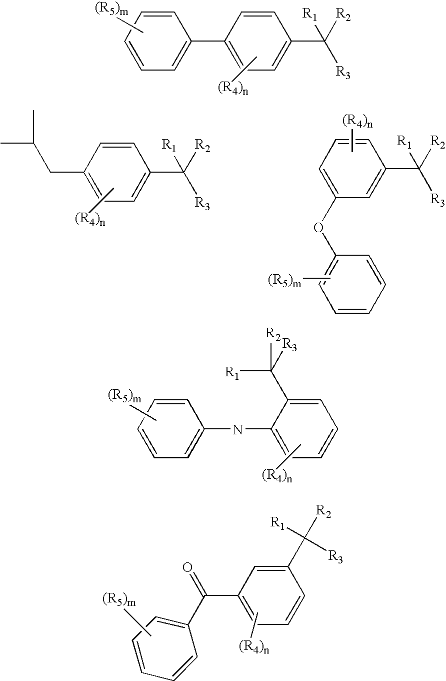
where R₁ is chosen from —CH₃, —CH₂CH₃, —CH₂CH₂CH₃, and —CH₂CH₂CH₂CH₃ (or can be taken together with R₂ to give a cyclopropyl ring, a cyclobutyl ring, a cyclopentyl ring, or a cyclohexyl ring);
R₂ is chosen from —CH₃, —CH₂CH₃, —CH₂CH₂CH₃, and —CH₂CH₂CH₂CH₃ (or can be taken together with R₁ to give a cyclopropyl ring, a cyclobutyl ring, a cyclopentyl ring, or a cyclohexyl ring);
R₃ is chosen from —COOH, —COOR₆, —CONH₂, —CONHR₆, —CONR₆R₇, —CONHSO₂R₆, tetrazolyl, and a —COOH bioisostere;
R₄ is chosen from —Cl, —F, —Br, —I, —CF₃, —OCF₃, —SCF₃, —OCH₃, —OCH₂CH₃, —CN, —CH═CH₂, —CH₂OH, and —NO₂;
R₅ is chosen from —Cl, —F, —Br, —I, —CF₃, —OCF₃, —SCF₃, —OCH₃, —OCH₂CH₃, —CN, —CH═CH₂, —CH₂OH, and —NO₂;
R₆ is chosen from —CH₃, —CH₂CH₃, —CH₂CH₂CH₃, and —CH₂CH₂CH₂CH₃;
R₇ is chosen from —CH₃, —CH₂CH₃, —CH₂CH₂CH₃, and —CH₂CH₂CH₂CH₃;
m is an integer chosen from 0, 1, 2, and 3;
n is an integer chosen from 0, 1, 2, and 3; and pharmaceutically acceptable salts thereof.

3. The co-formulation of claim 1 wherein said one or more Aβ₄₂ lowering agents is chosen from 2-methyl-2(2-fluoro-4′-trifluoromethylbiphen-4-yl) propionic acid; 2-methyl-2(2-fluoro-4′cyclohexyl biphen-4-yl) propionic acid; 1-(2-fluoro-4′-trifluoromethylbiphenyl-4-yl) cyclopropanecarboxylic acid; 1-(4′-cyclohexyl-2-fluorobiphenyl-4-yl) cyclopropanecarboxylic acid; 1-(4′-benzyloxy-2-fluorobiphenyl-4-yl) cyclopropanecarboxylic acid; 1-(2-fluoro-4′-isopropyloxybiphenyl-4-yl) cyclopropanecarboxylic acid; 1-(2-fluoro-3′-trifluoromethoxybiphenyl-4-yl) cyclopropanecarboxylic acid; 1-(2-fluoro-4′-trifluoromethoxybiphenyl-4-yl) cyclopropanecarboxylic acid; 1-(2-fluoro-3′-trifluoromethylbiphenyl-4-yl) cyclopropanecarboxylic acid; 1-(4′-cyclopentyl-2-fluorobiphenyl-4-yl) cyclopropanecarboxylic acid; 1-(4′-cycloheptyl-2-fluorobiphenyl-4-yl) cyclopropanecarboxylic acid; 1-(2′-cyclohexyl-2-fluorobiphenyl-4-yl) cyclopropanecarboxylic acid; 1-(2-fluoro-4′-hydroxybiphenyl-4-yl) cyclopropanecarboxylic acid; 1-[2-fluoro-4′-(tetrahydropyran-4-yloxy) biphenyl-4-yl]-cyclopropane-carboxylic acid; 1-(2,3′,4′-trifluorobiphenyl-4-yl) cyclopropanecarboxylic acid; 1-(3′,4′-dichloro-2-fluorobiphenyl-4-yl) cyclopropanecarboxylic acid; 1-(3′,5′-dichloro-2-fluorobiphenyl-4-yl) cyclopropanecarboxylic acid 1-(3′-chloro-2,4′-difluorobiphenyl-4-yl) cyclopropanecarboxylic acid; 1-(4-benzo[b]thiophen-3-yl-3-fluorophenyl) cyclopropanecarboxylic acid; 1-(2-fluoro-4′-prop-2-inyloxy-biphenyl-4-yl)-cyclopropanecarboxylic acid; 1-(4′-cyclohexyloxy-2-fluoro-biphenyl-4-yl)-cyclopropanecarboxylic acid; 1-[2-fluoro-4′-(tetrahydropyran-4-yl)-biphenyl-4-yl]-cyclopropanecarboxylic acid; 1-[2-fluoro-4′-(4-oxo-cyclohexyl)-biphenyl-4-yl]-cyclopropanecarboxylic acid; 2-(2″-fluoro-4-hydroxy-[1,1′:4′,1″]tert-phenyl-4″-yl)-cyclopropanecarboxylic acid; 1-[4′-(4,4-dimethylcyclohexyl)-2-fluoro[1,1′-biphenyl]-4-yl]-cyclopropane-carboxylic acid; 1-[2-fluoro-4′-[[4-(trifluoromethyl) benzoyl] ammino] [1,1′-biphenyl]-4-yl]-cyclopropanecarboxylic acid; 1-[2-fluoro-4′-[[4-(trifluoromethyl) cyclohexyl] oxy] [1,1′-biphenyl]-4-yl]-cyclopropanecarboxylic acid; 1-[2-fluoro-4′-[(3,3,5,5-tetramethylcyclohexyl) oxy] [1,1′-biphenyl]-4-yl]-cyclopropanecarboxylic acid; 1-[4′-[(4,4-dimethylcyclohexyl) oxy]-2-fluoro[1,1′-biphenyl]-4-yl]-cyclopropanecarboxylic acid; 1-(2,3′,4″-trifluoro[1,1′:4′,1″-tert-phenyl]-4-yl)-cyclopropanecarboxylic acid; 1-(2,2′,4″-trifluoro[1,1′:4′,1″-tert-phenyl]-4-yl)-cyclopropanecarboxylic acid; 1-(2,3′-difluoro-4″-hydroxy[1,1′:4′,1″-tert-phenyl]-4-yl)-cyclopropane-carboxylic acid; 1-(2,2′-difluoro-4″-hydroxy[1,1′:4′,1″-tert-phenyl]-4-yl)-cyclopropane-carboxylic acid; 2-(2-fluoro-3′,5′-bis(chloro)biphen-4-yl) propionic acid amide; 2-(2-fluoro-4′-trifluoromethylbiphen-4-yl) propionic acid; 2-(2-fluoro-3′-trifluoromethylbiphen-4-yl) propionic acid; 2-(2-fluoro-3′,5′-bis(trifluoromethyl)biphen-4-yl) propionic acid; 2-(4′-cyclohexyl-2-fluorobiphen-4-yl) propionic acid; 2-(2-Fluoro-1,1′-biphenyl-4-yl)-2-methylpropanoic acid; 2-Methyl-2-(3-phenoxy-phenyl)-propionic acid; 2-(4-Isobutyl-phenyl)-2-methyl-propionic acid; 2-(6-Chloro-9H-carbazol-2-yl)-2-methyl-propionic acid; 2-[1-(4-Chloro-benzoyl)-5-methoxy-2-methyl-1H-indol-3-yl]-2-methyl-propionic acid; 5-[1-(2-Fluoro-biphenyl-4-yl)-1-methyl-ethyl]-2H-tetrazole, and pharmaceutically acceptable salts thereof.

4. The co-formulation of claim 1 wherein said one or more Aβ₄₂ lowering agents is chosen from (R)-2-(2-fluoro-4-biphenylyl)propionic acid, 5[1-(2-Fluoro-biphenyl-4-yl)-1-methyl-ethyl]-2H-tetrazole, 2-(4-isobutyl-phenyl)-2-methyl propionic acid, 2-(2-fluoro-1,1′-biphenyl-4-yl)-2-methylpropionic acid, and pharmaceutically acceptable salts thereof.

5. The co-formulation of claim 1 wherein said one or more Aβ₄₂ lowering agents is (R)-2-(2-fluoro-4-biphenylyl)propionic acid or a pharmaceutically acceptable salt thereof.

6. The co-formulation of claim 1 wherein said one or more hormonal modulating agents are chosen from steroids, non-steroids, estrogens, antiandrogens, antiestrogens, progestins, aromatase inhibitors, inhibitors of sex steroid biosynthesis, vitamin D3, vitamin D3 derivatives, vitamin D3 analogues, vitamin D, prolactin secretion inhibitors, steroidal anti-inflammatory agents, and pharmaceutically acceptable salts thereof.

7. The co-formulation of claim 1 wherein said one or more hormonal modulating agents is steroidal anti-inflammatory agent chosen from budesonide, pregnenolone, prednisone, prednisolone, methylprednisolone, triamcinolone, dexamethasone, betamethasone, parametasone, cortisone, and hydrocortisone.

8. The co-formulation of claim 1 wherein said one or more hormonal modulating agents is a steroidal anti-inflammatory agent chosen from budesonide, pregnenolone, prednisone, prednisolone, methylprednisolone, triamcinolone, dexamethasone, betamethasone, parametasone, cortisone, and hydrocortisone and said one or more Aβ₄₂ lowering agents is (R)-2-(2-fluoro-4-biphenylyl)propionic acid or a pharmaceutically acceptable salt thereof.

9. The co-formulation of claim 1 wherein said one or more hormonal modulating agents are chosen from a glucocorticoid, an estrogen, and an androgen.

10. The co-formulation of claim 1 wherein said one or more hormonal modulating agents is chosen from vitamin D3, vitamin D3 analogues, and vitamin D3 derivatives.

11. The co-formulation of claim 1 wherein said one or more hormonal modulating agents is calcitriol.

12. The co-formulation of claim 1 wherein said one or more hormonal modulating agents is a steroid chosen from alclometasone, amcinonide, beclomethasone, betamethasone, clobetasol, clocortolone, hydrocortisone, cortisol, cortisone, desonide, desoximetasone, dexamethasone, diflorasone, fludrocortisone, flunisolide, fluocinolone, fluocinonide, fluorometholone, flurandrenolide, halcinonide, medrysone, methylprednisolone, mometasone, paramethasone, prednisolone, prednisone, triamcinolone, and pharmaceutically acceptable salts thereof.

13. The co-formulation of claim 1 wherein said one or more hormonal modulating agents is an antiandrogen

14. The co-formulation of claim 13 wherein said antiandrogen is chosen from flutamide, bicalutamide, and nilutamide.

15. The co-formulation of claim 1 wherein said one or more hormonal modulating agents is an antiestrogen

16. The co-formulation of claim 15 wherein said antiestrogen is chosen from tamoxifen, (1RS,2RS)-4,4′-diacetoxy-5,5′-difluoro-(1-ethyl-2-methylene)di-m-phenylen-ediacetate, 6α-chloro-16α-methyl-pregn-4-ene-3,20-dione, 6-chloro-17-hydroxypregna-1,4,6-triene-3,20-dione, 17-hydroxy-6-methyl-19-norpregna-4,6-diene-3,20-dione, 1-[2-[4-[1-(4-methoxyphenyl)-2-nitro-2-phenylethenyl)phenoxy]ethyl]-pyrrolidine, substituted aminoalkoxyphenylalkenes, 3,4-dihydro-2-(p-methoxyphenyl) -1-naphthyl p-[2-(1-pyrrolidinyl)ethoxy]phenyl ketone, 1-[4′-(2-phenyl)-bl-(3′-hydroxyphenyl)-2-phenyl-but-1-ene, [6-hydroxy-2-(p-hydroxyphenyl-)-benzo(b)thien-3yl]-[2-(1-pyrrolidinyl)-ethoxy phenyl]ketone, [6-hydroxy-2-(4-hydroxyphenyl)benzo(b)thien-3-yl]-[4-(2-(1-piperdinyl)ethoxy)phenyl]methanone, meso-3,4-bis(3′-hydroxyphenyl) hexane, 7α-substituents of estradiol, and pharmaceutically acceptable salts thereof.

17. The co-formulation of claim 1 wherein said one or more hormonal modulating agents is chosen from 4-methyl-2[4-[2-(1-piperidinyl)ethoxy]phenyl]-7-(pivaloyloxy)-3-[4-(pivaloyloxy)phenyl]-2H-1-benzopyran, (5,6,7,8-tetrahydro-6-phenyl-5-(4-(2-(1-pyrrolidinyl)ethoxy)phenyl-(5R- cis)-2-naphthalenol, (S—(R*,R*))-2,3-dihydroxybutanedioate), 2-methoxyestradiol, ((2-(4-methoxyphenyl)-3-(4-(2-(1-piperidinyl)ethoxy)phenoxy)-benzo(b)thi-ophene-6-ol, hydrochloride), (4,4′-dyhydroxybenzophenone-2,4-dinitrophenylhydrazone), ((7α,17β)-7-[9-[(4,4,5,5,5-pentafluoropentyl)sulfinyl]no-nyl]-estra-1,3,5(10)-triene-3,17-diol), and pharmaceutically acceptable salts thereof.

18. The co-formulation of claim 1 wherein said one or more hormonal modulating agents is an inhibitor of sex steroid biosynthesis

19. The co-formulation of claim 1 wherein said inhibitor of sex steroid biosynthesis agents is chosen from aminoglutethimide, ketoconazole, 4-hydroxyandrostenedione, atamestane, exemestane, anastrazole, fadrozole, finrozole, letrozole, vorozole, YM-511, and pharmaceutically acceptable salts thereof.

20. The co-formulation of claim 1 wherein said one or more hormonal modulating agents is an inhibitors of 3β-hydroxysteroid or Δ5-Δ4-isomerase activity.

21. The co-formulation of claim 1 wherein said inhibitor of 3β-hydroxysteroid or Δ5-Δ4-isomerase activity is chosen from trilostane, eposlane or 4-MA.

23. The co-formulation of claim 1 wherein said one or more hormonal modulating agents is a vitamin D3 related compound.

24. The co-formulation of claim 1 wherein said vitamin D3 related compound is chosen from calciol (or cholecalciferol), ercalciol (or ergocalciferol), calcidiol, (1S)--hydroxycalciol, (24R)-hydroxycalcidiol, calcitriol, calcitetrol, 25-fluorocalciol, ercalcidiol, ercalcitriol, ertacalciol, tacalciol, (5E)-isocalciol, 22,23-dihydroercalciol (or (24S)-methylcalciol), (5E)-(10S)-10,19-dihydroercalciol, (6Z)-tacalciol, (24S)-ethylcalciol, (22E)-(24R)-ethyl-22,23-didehydrocalciol, 25-Dihydroxy-20epi-22-oxa-24,26,27-trisho-mocholecalciferol (KH 1060), 1,25-Dihydroxy-22E,24E-diene-24,26,27-trishomocholecalciferol (EB 1039), 1,25-Dihydroxy-16-ene-24-oxo-19-norcholecalciferol, and pharmaceutically acceptable salts thereof.

25. A method of treating an individual with a combination of an Aβ₄₂ lowering agent and a hormonal modulating agent, said method comprising:
(a) identifying an individual in need of such treatment; and
(b) administering to said individual a therapeutically effective amount of an Aβ₄₂ lowering agent and a hormonal modulating agent.

26. The method of claim 25, wherein said individual in need of treatment has a neurodegenerative disorder.

27. The method of claim 25, wherein said individual in need of treatment has Alzheimer's disease.

28. The method of claim 25, wherein said individual in need of treatment has dementia.

27. The method of claim 25, wherein said individual in need of treatment has mild cognitive impairment.

28. The method of claim 25, wherein said individual in need of treatment has mild Alzheimer's disease.

29. The method of claim 25, wherein said individual in need of treatment has cancer or is seeking prophylaxis against cancer.

30. The method of claim 25 wherein said individual has prostate cancer, or is at medium to high risk of having prostate cancer recurrence after a radiotherapy and/or surgery.

31. The method of claim 25, wherein said administering comprises co-administration of the Aβ₄₂ lowering agent and hormonal modulating agent.

32. The method of claim 25, wherein said Aβ₄₂ lowering agent and hormonal modulating agent are not co-administered.

33. The method of claim 25, wherein said Aβ₄₂ lowering agent and hormonal modulating agent are co-formulated.

34. The method of claim 25, wherein said Aβ₄₂ lowering agent is chosen from (R)-2-(2-fluoro-4-biphenylyl)propionic acid, 5[1-(2-Fluoro-biphenyl-4-yl)-1-methyl-ethyl]-2H-tetrazole, 2-(4-isobutyl-phenyl)-2-methyl propionic acid, 2-(2-fluoro-1,1′-biphenyl-4-yl)-2-methylpropionic acid, and pharmaceutically acceptable salts thereof.

35. The method of claim 25, wherein said Aβ₄₂ lowering agent is (R)-2-(2-fluoro-4-biphenylyl)propionic acid or a pharmaceutically acceptable salt thereof.

36. The method of claim 25, wherein said hormonal modulating agent is a steroidal anti-inflammatory agent.

37. The method of claim 36, wherein said steroidal anti-inflammatory agent is chosen from budesonide, pregnenolone, prednisone, prednisolone, methylprednisolone, triamcinolone, dexamethasone, betamethasone, parametasone, cortisone, and hydrocortisone.
